(19) United States
(12) Patent Application Publication
(10) Pub. No.: US 2022/0289684 A1
(43) Pub. Date: Sep. 15, 2022

(54) BENZIMIDAZOLE SUBSTITUTION-BASED PHENYL N-BUTYRAMIDE COMPOUND AND PREPARATION METHOD THEREFOR

(71) Applicants: TOPHARMAN SHANGHAI CO., LTD., Shanghai (CN); SHANGHAI INSTITUTE OF MATERIAMEDICA,CHINESE ACADEMY OF SCIENCES, Shanghai (CN); TOPHARMAN TANCHENG CO.,LTD., Shandong (CN)

(72) Inventors: Jingshan SHEN, Shanghai (CN); Changliang SUN, Shanghai (CN); Fuqiang ZHU, Shanghai (CN); Junchi ZHANG, Shanghai (CN); Rui LI, Shanghai (CN)

(21) Appl. No.: 17/637,637

(22) PCT Filed: Aug. 27, 2020
(86) PCT No.: PCT/CN2020/111668

§ 371 Date: May 27, 2022

(30) Foreign Application Priority Data
Aug. 29, 2019 (CN) 201910807292.2
Aug. 25, 2020 (CN) 202010863046.1

(51) Int. Cl. C07D 235/18; C07C 237/34

(52) U.S. Cl. CPC C07D 235/18; C07C 237/34

(57) ABSTRACT

The present disclosure relates to a benzimidazole-substituted phenyl-n-butyramide-based compound and the preparation method thereof. The method of the present disclosure avoids nitration reaction and polyphosphoric acid cyclization reaction, and avoids the generation of a large amount of waste acid reaction solution from the source. The synthesis method embodied in the invention has the advantages of simplicity and high efficiency, mild conditions and few pollutants, etc., and is suitable to be developed as a green and sustainable production process.

This disclosure claims priority to CN patent application No. 2019108072922 filed on Aug. 29, 2019, and 2020108630461 filed on Aug. 25, 2020.

### TECHNICAL FIELD

The present disclosure relates to a pharmaceutical compound and a preparation method thereof Specifically, the present disclosure relates to benzimidazole-substituted phenyl-n-butyramide-based compounds and preparation methods thereof

### BACKGROUND ART

Telmisartan, which is a novel non-peptide angiotensin II (ATII type) receptor antagonist, is a new hypotensive drug for clinical treatment. Telmisartan was first developed by Boehringer Ingelheim and was first marketed in the United States in March 1999, and then in many other countries around the world. The chemical name for Telmisartan is 4′-[(1,4′-dimethyl-2′-propyl[2,6′-bis-1H-benzimidazol]-1′-yl)methyl]-[1,′-biphenyl]-2-carboxylic acid with the following structure:

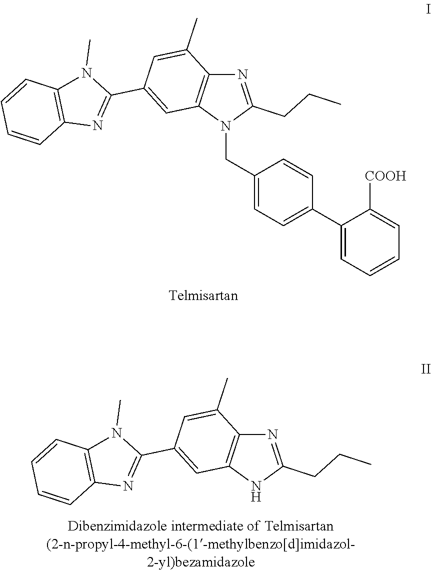 [SMILES: CCCc1nc2c(C)cc(-c3nc4ccccc4n3C)cc2n1Cc1ccc(-c2ccccc2C(=O)O)cc1 ; CCCc1nc2c(C)cc(-c3nc4ccccc4n3C)cc2[nH]1]

The molecular structure of telmisartan contains two connected benzimidazole rings, and the construction of the bis-benzimidazole ring structure is the strategic focus in the synthesis of telmisartan. Among many bisbenzimidazole intermediates, the most representative is 2-n-propyl-4-methyl-6-(1′-methylbenzo[d]imidazol-2-yl)benzimidazole, which is the most involved intermediate in the known synthetic route of telmisartan.

In the currently known synthetic routes, the mainly adopted synthetic strategy is to first construct the benzimidazole ring located in the middle of the structure, and then construct the other benzimidazole ring located at the end of the structure. The benzimidazole ring located in the middle of the structure is generally constructed by using a substituted n-butyranilide as a raw material, introducing a nitro group by nitration at ortho-position of the n-butyramide group of the aromatic ring, reducing the nitro group to an amino group, and cyclizing by condensation. There are still many problems in the synthetic route under this strategy, such as the safety of nitration reaction and the disposal of nitrification waste liquid, a large amount of waste acid liquid generated during formation of the second imidazole ring in polyphosphoric acid or a strong acid, and the disposal of the waste for neutralizing the waste acid, etc.

Therefore, it is particularly important to discover and develop a new synthetic route and process conditions for the bisbenzimidazole intermediate of telmisartan that is safer, more environmentally friendly, simpler, more efficient, milder, less costly, and suitable for industrial production. At the same time, the new method must also meet the requirements of the ESH management system, conform to the higher pursuit and concept of safe, environmentally friendly and green synthesis, and be suitable for developing a green and sustainable production process.

### SUMMARY OF THE INVENTION

An object of the present disclosure is to provide a benzimidazole-substituted phenyl-n-butyramide-based compound or a salt thereof.

Another object of the present disclosure is to provide a method for preparing the benzimidazole-substituted phenyl-n-butyramide-based compound.

According to an embodiment of the present disclosure, provided is a benzimidazole-substituted phenyl-n-butyramide-based compound of formula III or a salt thereof:

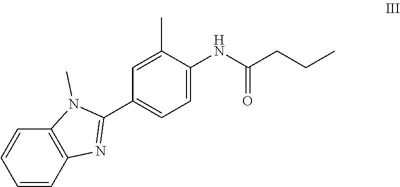 [SMILES: CCCC(=O)Nc1ccc(-c2nc3ccccc3n2C)cc1C]

In particular, the salt is III·HX, a salt formed by the compound of formula III with HX which is an acid selected from the group consisting of hydrochloric acid, hydrobromic acid, sulfuric acid, phosphoric acid, formic acid and acetic acid.

According to another embodiment of the present disclosure, provided is a method for preparing the benzimidazole-substituted phenyl-n-butyramide-based compound of formula III, wherein the method is one selected from the group consisting of the following methods:

Method One: prepared by reacting the compound of formula IV with a methylating reagent,

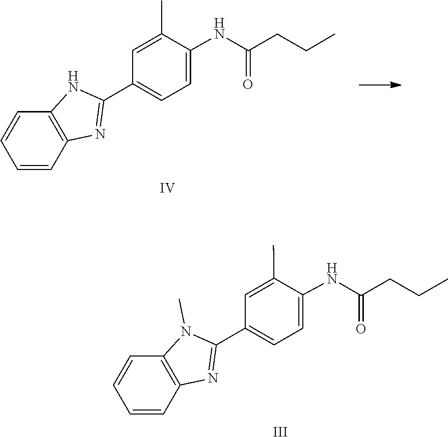 [SMILES: CCCC(=O)Nc1ccc(-c2nc3ccccc3[nH]2)cc1C ; "IV" ; CCCC(=O)Nc1ccc(-c2nc3ccccc3n2C)cc1C ; "III"]

In particular, the methylating agent is selected from the group consisting of methyl iodide, dimethyl sulfate and dimethyl carbonate;

In particular, the reaction is carried out in the presence of a basic reagent in a solvent,

In particular, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;

In particular, the solvent is one selected from the group consisting of tetrahydrofuran, dioxane, ethylene glycol dimethyl ether, methanol, ethanol, isopropanol, n-butanol, tert-butanol, ethylene glycol, acetone, acetonitrile, N,N-dimethylformamide, N,N-dimethylacetamide, N-methylpyrrolidone, dimethyl sulfoxide and water; or a mixture thereof;

Method Two: prepared by the compound of formula V-1,

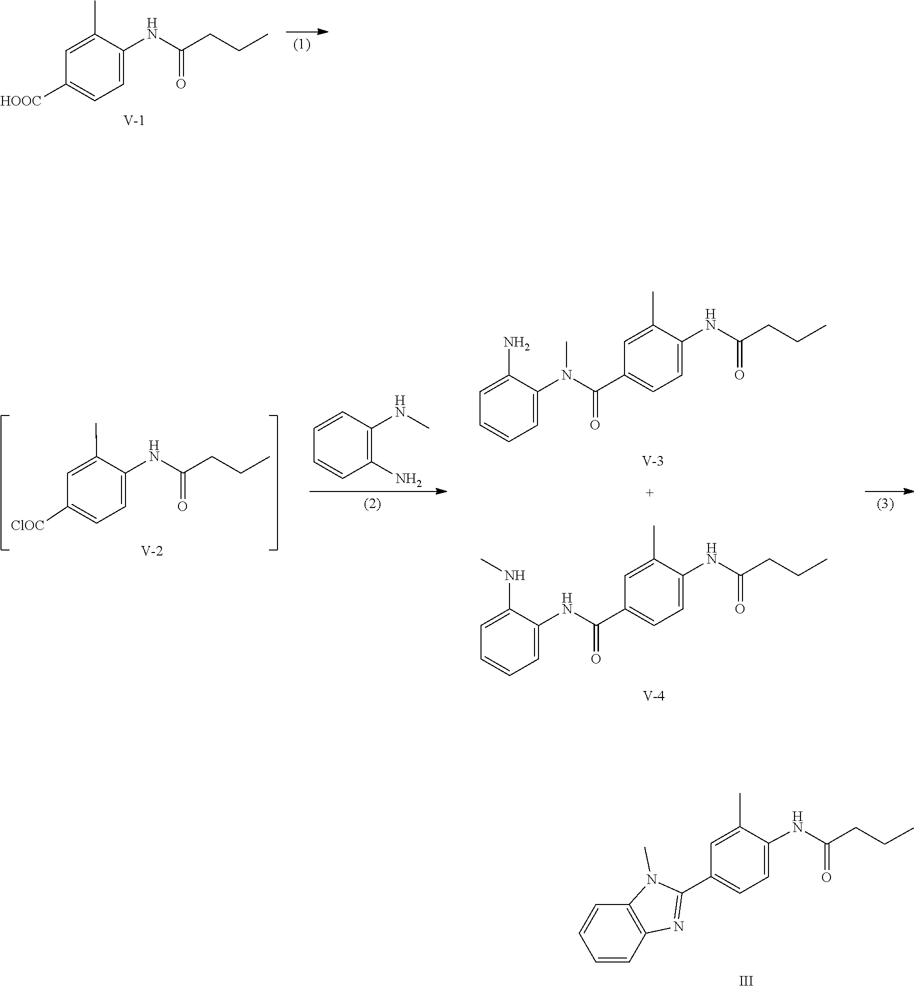

Step (1), preparing 3-methyl-4-n-butyrylaminobenzoyl chloride of formula V-2 through chlorination reaction by using 3-methyl-4-n-butyrylaminobenzoic acid as a starting material;

Step (2), reacting the compound of formula V-2 with N-methyl-o-phenylenediamine to obtain the compounds of formulae V-3 and V-4;

Step (3), conducting a condensation reaction of the compounds of formulae V-3 and V-4 in the presence of an acidic reagent, a basic reagent or a condensation reagent, to obtain the compound of formula III;

Particularly in the step (1), the chlorinating reagent used in the chlorination reaction is one selected from the group consisting of thionyl chloride, phosphorus oxychloride, phosphorus pentachloride, oxalyl chloride, phosgene, and bis(trichloromethyl) carbonate (triphosgene), or a mixture thereof;

Particularly in the step (1), the chlorination reaction is carried out in a solvent, in particular, the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, and methyl tert-butyl ether, or a mixture thereof; preferably is methylene chloride;

Particularly in the step (2), a basic reagent is used as an acid binding agent, in particular, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;

In particular, the step (2) is carried out in a solvent, in particular, the solvent used is one selected from the group consisting of tetrahydrofuran, dioxane, 2-methyltetrahydrofuran, ethylene glycol dimethyl ether, methyl tert-butyl ether, toluene, xylene, dichloromethane, chloroform, acetonitrile, acetone, pyridine, N,N-dimethylformamide, N-methylpyrrolidone, dimethyl sulfoxide, and water, or a mixture thereof;

In particular, the step (1) and the step (2) are carried out in one-pot method, that is, the compound of formula V-2 prepared in the step (1) is directly subjected to the reaction of the step (2) without separation;

In particular, in the step (3), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;

Particularly in the step (3), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof

Particularly in the step (3), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;

In particular, in the step (3), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof Alternatively, a solvent-free reaction condition is used.

Method Three: prepared by the compound of formula V-1,

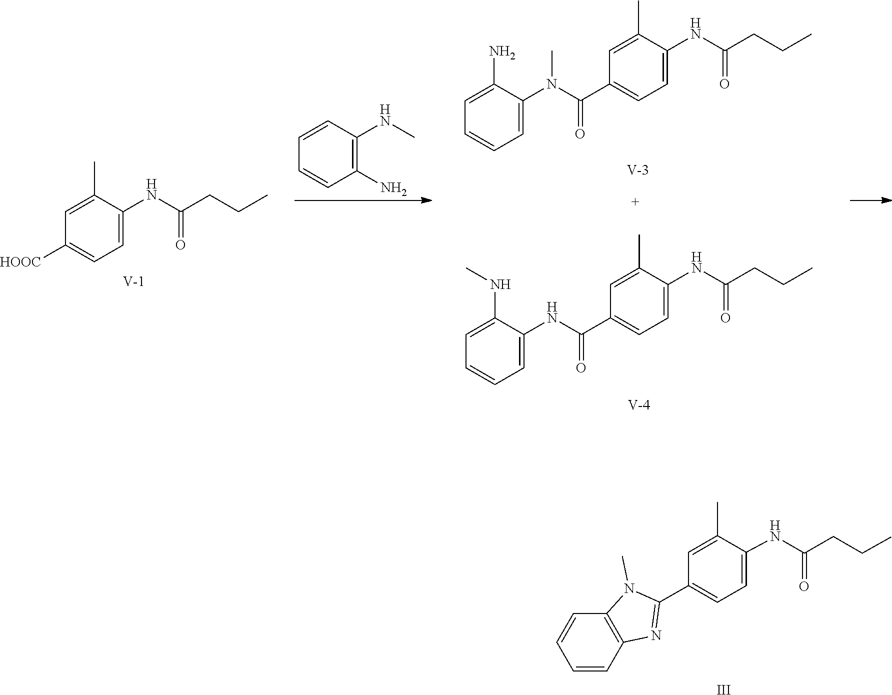

Step (1), reacting 3-methyl-4-n-butyrylaminobenzoic acid with N-methyl-o-phenylenediamine in the presence of an acidic reagent or a condensation reagent to obtain the compounds of formulae V-3 and V-4;

Step (2), reacting the compounds of formulae V-3 and V-4 in the presence of the acidic reagent or the condensation reagent in the step (1), or a basic reagent, to obtain the compound of formula III;

In particular, the step (1) and the step (2) are carried out in one-pot method, that is, in the presence of the acidic reagent or the condensation reagent, the compound of formulae V-3 and V-4 prepared in the step (1) continually react under the reaction condition of step (1) without separation to obtain the compound of formula III;

In particular, in the step (1), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;

Particularly in the step (1), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole,

O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;

In particular, in the step (2), the acidic reagent or the condensation reagent used is the same as defined in the step (1).

Particularly in the step (2), the basic reagent used is one selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof

In particular, in the step (1) and the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof Alternatively, a solvent-free reaction condition is used.

Method Four: prepared by the compound of formula V-6 or V-7,

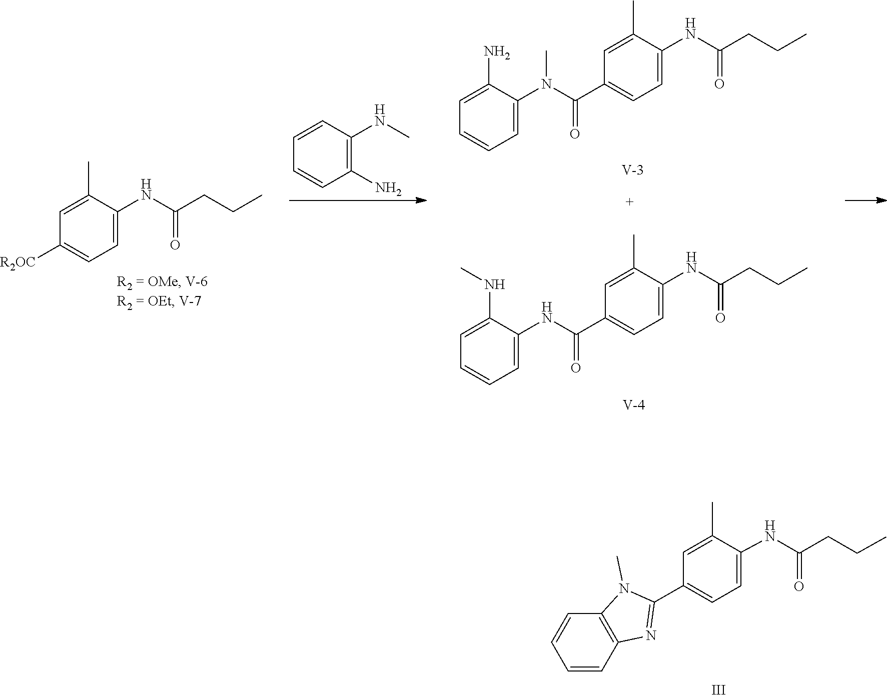

Step (1), reacting methyl 3-methyl-4-n-butyrylaminobenzoate or ethyl 3-methyl-4-n-butyrylaminobenzoate, with N-methyl-o-phenylenediamine in the presence of an acidic reagent or a basic reagent to obtain the compounds of formula V-3 and V-4;

Step (2), reacting the compounds of formulae V-3 and V-4 in the presence of the acidic reagent or the basic reagent in the step (1), or a condensation reagent, to obtain the compound of formula III;

In particular, the step (1) and the step (2) are carried out in one-pot method, that is, in the presence of the acidic reagent or the basic reagent, the compound of formulae V-3 and V-4 prepared in the step (1) continually react under the reaction condition of step (1) without separation to obtain the compound of formula III;

In particular, in the step (1), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;

Particularly in the step (1), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof

In particular, in the step (2), the acidic reagent or the basic reagent used is the same as defined in the step (1).

Particularly in the step (2), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof; In particular, in the step (1) and the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof Alternatively, a solvent-free reaction condition is used.

Method Five: prepared by the compound of formula VI,

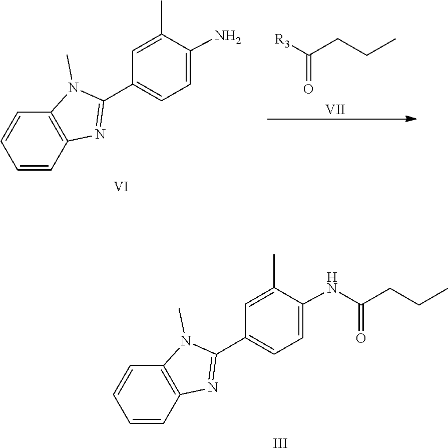

reacting 2-methyl-4-(1-methyl-1H-benzo[d]imidazol-2-yl) aniline with the compound of formula VI to obtain the compound of formula III;

Method Six: prepared by the compound of formula VIII,

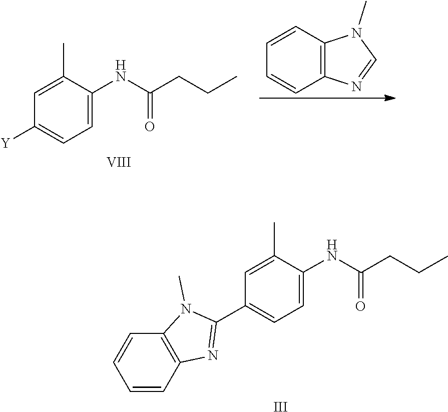

wherein, Y is Cl, Br and I,

reacting the compound of formula VIII with N-methylbenzimidazole in the presence of a basic reagent to obtain the compound of formula III,

In particular, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, sodium acetate, potassium acetate, magnesium acetate, sodium pivalate, potassium pivalate, magnesium pivalate, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;

In particular, the reaction is carried out in the presence of a transition metal compound and the complex thereof, the transition metal compound is one selected from the group consisting of cuprous chloride, cuprous bromide, cuprous iodide, cuprous oxide, cuprous cyanide, cuprous acetate, copper chloride, copper bromide, copper oxide, copper acetate, copper sulfate, copper nitrate, palladium chloride, palladium acetate, palladium trifluoroacetate, palladium trifluoromethanesulfonate, bis (dibenzylideneacetone)palladium, tris(dibenzylideneacetone)dipalladium, tetrakis(triphenylphosphine)palladium, nickel dichloride, nickel acetate, bis(acetylacetone)nickel, nickel trifluoroacetate, nickel trifluoromethanesulfonate, and bis(1,5-cyclooctadiene)nickel, or a mixture thereof; the ligand used for the transition metal compound is one selected from the group consisting of ethylenediamine, N-methylethylenediamine, N-butyl ethylenediamine, N,N′-dimethylethylenediamine, N,N-dimethylethylenediamine, trimethylethylenediamine, tetramethylethylenediamine, (cis)-1,2-cyclohexanediamine, (trans)-1,2-cyclohexanediamine, 1,2-cyclohexanediamine racemate, (trans)-N,N′-dimethyl-1,2-cyclohexanediamine, (trans)-N,N′-diethyl-1,2-cyclohexanediamine, (trans)-N,N′-diisopropyl-1,2-cyclohexanediamine, 2,2′-bipyridine, 1,10-phenanthroline, 2,9-dimethyl-1,10-phenanthroline, 3,4,7, 8-tetramethyl-1,10-phenanthroline, 4,7-diphenyl-1,10-phenanthroline, triphenylphosphine, tricyclohexylphosphine, tri-tert-butylphosphine, 1,2-bis(diphenylphosphine)ethane, 1,2-bis(diphenylphosphine)propane, 1,1′-bis(di-phenylphosphine)ferrocene, 4,5-bis(diphenylphosphine)-9,9-dimethylxanthene and 1,1′-binaphthyl-2,2′-bisdiphenylphosphine, or a mixture thereof, In particular, the reaction is carried out in a solvent, the solvent is one selected from the group consisting of dioxane, 2-methyltetrahydrofuran, ethylene glycol dimethyl ether, toluene, xylene, acetonitrile, acetone, ethanol, isopropanol, n-butanol, tert-butanol, ethylene glycol, pyridine, N,N-dimethylformamide, N,N-dimethylacetamide, N-methylpyrrolidone, dimethyl sulfoxide, and water, or a mixture thereof.

According to one embodiment of the present disclosure, in method Five, is the compound of formula VI is prepared by the following method:

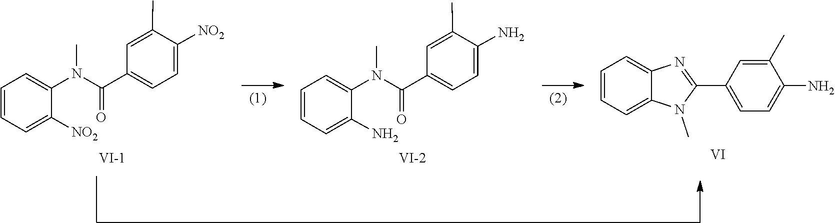

Step (1), obtaining the compound of formula VI-2 from the compound of formula VI-1 under reducing conditions;

Step (2), obtaining the compound of formula VI from the compound of formula VI-2 in the presence of an acidic reagent, a basic reagent, or a condensation reagent;

Alternatively, obtaining directly the compound of formula VI from the compound of formula VI-1 by adding an acidic reagent or a basic reagent in the reduction system of step (1); in particular, the acidic reagent or the basic reagent used in the reaction process is the same as defined in the step (1) of the above methodFour;

Particularly in the step (1), the reducing agent used in the reducing conditions is a nitro reducing agent, which is selected from the group consisting of hydrogen, metal reducing agents, metal chlorides, complex hydrides, sulfur-containing reducing agents, and so on, wherein the hydrogen reduction is conducted with the addition of a catalyst, the catalyst used is selected from the group consisting of Cu, Ni, Pd, Pt, Ru, Rh and their oxides, hydroxides, chlorides, complexes formed with carbon or corresponding organometallic complexes; the metal reducing agent is selected from the group consisting of iron powder and zinc powder; the metal chloride is selected from the group consisting of stannous chloride dihydrate, and titanium trichloride; the complex hydride is selected from the group consisting of lithium aluminum hydride; the sulfur-containing reducing agent is selected from the group consisting of sodium hydrosulfide, sodium sulfide, ammonium sulfide, sodium sulfite, sodium bisulfite, and sodium dithionite;

In particular, the step (1) is carried out in a solvent, the solvent is one selected from the group consisting of methanol, ethanol, propanol, isopropanol, n-butanol, tert-butanol, ethylene glycol, dioxane, 2-methyl tetrahydrofuran, ethylene glycol dimethyl ether, toluene, xylene, acetone, ethyl acetate, n-butyl acetate, N,N-dimethylformamide, N,N-dimethylacetamide, dimethylsulfoxide, formic acid, acetic acid, hydrochloric acid, sulfuric acid and water, or a mixture thereof;

In particular, the acidic reagent, the basic reagent or the condensation reagent in the step (2) is the same as defined in the step (2) of method Four;

In particular, in the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof Alternatively, a solvent-free reaction condition is used.

According to an embodiment of the present disclosure, the method Five is conducted according to one of the following three methods:

Method (a), when R₃ is chlorine, or bromine, or n-butyryloxy, reacting the compound of formula VI with n-butyryl chloride, or n-butyryl bromide, or n-butyric anhydride to prepare the compound of formula III,

In particular, method (a) is conducted in presence of a basic reagent, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;

In particular, method (a) is carried out in a solvent, in particular, the solvent used is one selected from the group consisting of tetrahydrofuran, dioxane, 2-methyltetrahydrofuran, ethylene glycol dimethyl ether, methyl tert-butyl ether, toluene, xylene, dichloromethane, chloroform, acetonitrile, acetone, pyridine, N,N-dimethylformamide, N-methylpyrrolidone, dimethyl sulfoxide, and water, or a mixture thereof;

Method (b), when R₃ is methoxy or ethoxy, reacting the compound of formula VI with methyl n-butyrate or ethyl n-butyrate to prepare the compound of formula III,

In particular, method (b) is carried out in the presence of an acidic reagent or a basic reagent,

Particularly in method (b), the acidic reagent used is one selected from the group consisting of hydrochloric acid, sulfuric acid, nitric acid, phosphoric acid, polyphosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof,

Particularly in the step (b), when using an acidic reagent, the reaction is carried out in a solvent or using solvent-free reaction conditions, in particular, when using an acidic reagent, the solvent used is one selected from the group consisting of dichloromethane, chloroform, benzene, toluene, xylene, methanol, ethanol, isopropanol, n-butanol, tert-butanol, and ethylene glycol, or a mixture thereof,

Particularly in method (b), the basic reagent used is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof, preferably is sodium methoxide,

Particularly in the method (b), when using a basic reagent, the reaction is carried out in a solvent, in particular, when using a basic reagent, the solvent used is one selected from the group consisting of dichloromethane, chloroform, benzene, toluene, xylene, methanol, ethanol, isopropanol, n-butanol, tert-butanol, ethylene glycol, N,N-dimethylformamide, N,N-dimethylacetamide, and N-methylpyrrolidone, or a mixture thereof;

Method (c), when R₃ is hydroxyl, reacting the compound of formula VI with n-butyric acid in the presence of an acidic reagent or a condensation reagent to prepare the compound of formula III,

Particularly in method (c), the acidic reagent used is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof,

Particularly in method (c), when using an acidic reagent, the reaction is carried out in a solvent or using solvent-free reaction conditions, in particular, when using an acidic reagent, the solvent is one selected from the group consisting of dichloromethane, chloroform, benzene, toluene, xylene, methanol, ethanol, isopropanol, n-butanol, tert-butanol, and ethylene glycol, or a mixture thereof,

Particularly in method (c), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;

Particularly in the method (c), when using a condensation reagent, the reaction is carried out in a solvent, in particular, when using a condensation reagent, the solvent used is one selected from the group consisting of dichloromethane, chloroform, benzene, toluene, xylene, tetrahydrofuran, dioxane, ethylene glycol dimethyl ether, acetone, acetonitrile, N,N-dimethylformamide, N,N-dimethylacetamide, and N-methylpyrrolidone, or a mixture thereof.

According to one embodiment of the present disclosure, wherein the compound of formula IV is prepared by one of the following methods:

Method I: prepared by the compound of formula V-1,

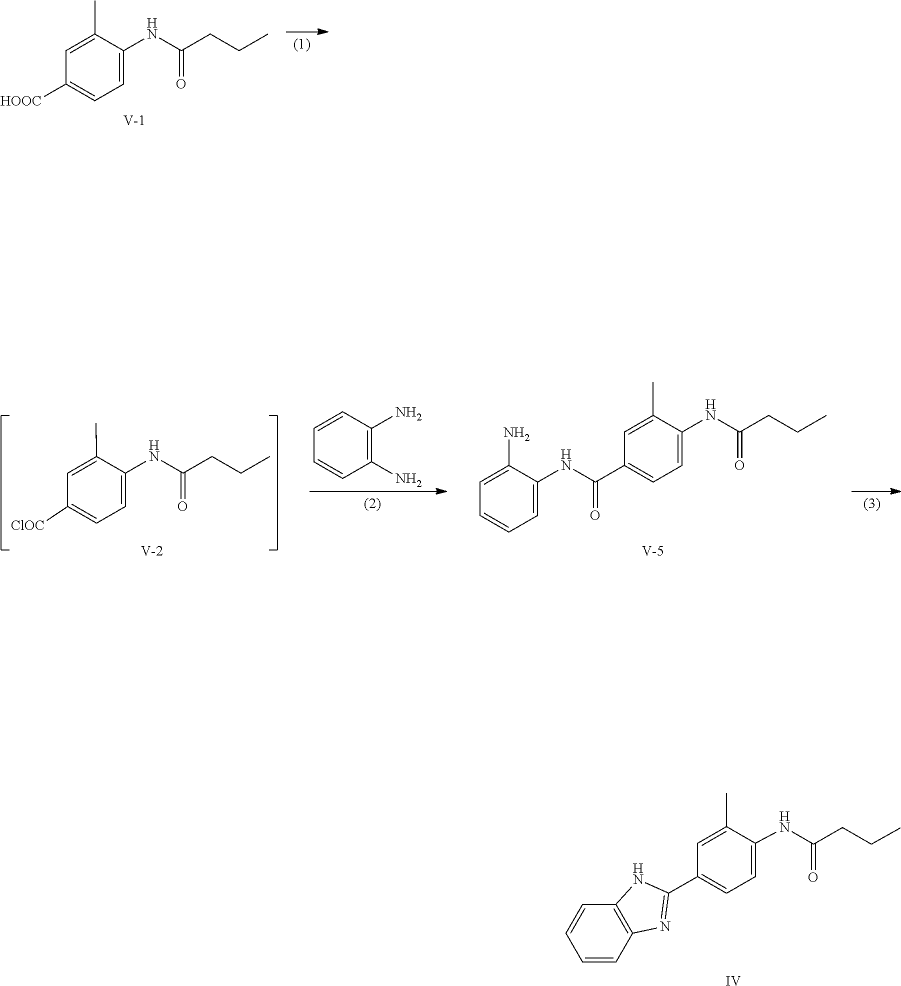

Step (1), preparing 3-methyl-4-n-butyrylaminobenzoyl chloride through chlorination reaction by using 3-methyl-4-n-butyrylaminobenzoic acid as a starting material,

Step (2), reacting the produced compound of formula V-2 with o-phenylenediamine to obtain the compound of formula V-5;

Step (3), conducting a condensation reaction of the compound of formula V-5 in the presence of an acidic reagent, a basic reagent or a condensation reagent, to obtain the compound of formula IV;

Particularly in the step (1), the chlorinating reagent used in the chlorination reaction is one selected from the group consisting of thionyl chloride, phosphorus oxychloride, phosphorus pentachloride, oxalyl chloride, phosgene, and bis(trichloromethyl) carbonate (triphosgene), or a mixture thereof;

Particularly in the step (1), the chlorination reaction is carried out in a solvent, in particular, the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, and methyl tert-butyl ether, or a mixture thereof; preferably is methylene chloride;

Particularly in the step (2), a basic reagent is used as an acid binding agent, in particular, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;

In particular, the step (2) is carried out in a solvent, in particular, the solvent used is one selected from the group consisting of tetrahydrofuran, dioxane, 2-methyltetrahydrofuran, ethylene glycol dimethyl ether, methyl tert-butyl ether, toluene, xylene, dichloromethane, chloroform, acetonitrile, acetone, pyridine, N,N-dimethylformamide, N-methylpyrrolidone, dimethyl sulfoxide, and water, or a mixture thereof;

In particular, the step (1) and the step (2) are carried out in one-pot method, that is, the compound of formula V-2 prepared in the step (1) is directly subjected to the reaction of the step (2) without separation;

In particular, in the step (3), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;

Particularly in the step (3), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof.

Particularly in the step (3), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof; In particular, in the step (3), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof Alternatively, a solvent-free reaction condition is used.

Method II: prepared by the compound of formula V-1,

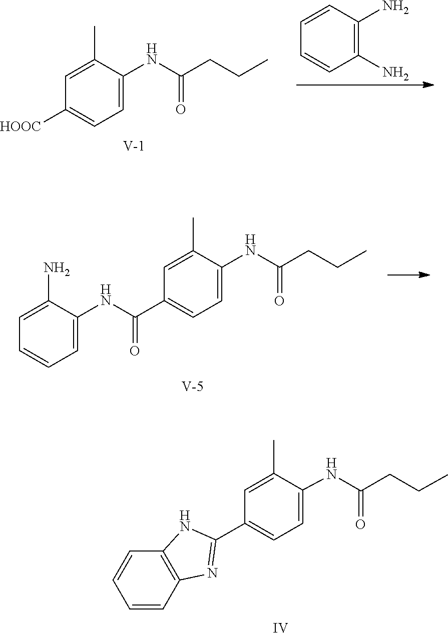

Step (1), reacting 3-methyl-4-n-butyrylaminobenzoic acid with o-phenylenediamine in the presence of an acidic reagent or a condensation reagent to obtain the compound of formula V-5;

Step (2), reacting the compound of formula V-5 in the presence of the acidic reagent or the condensation reagent in the step (1), or a basic reagent, to obtain the compound of formula IV;

In particular, the step (1) and the step (2) are carried out in one-pot method, that is, the compound of formula V-5 prepared in the step (1) continually reacts under the reaction condition of step (1) without separation to obtain the compound of formula IV;

In particular, in the step (1), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;

Particularly in the step (1), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;

In particular, in the step (2), the acidic reagent or the condensation reagent used is the same as defined in the step (1).

Particularly in the step (2), the basic reagent used is one selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof.

In particular, in the step (1) and the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof Alternatively, a solvent-free reaction condition is used.

Method III:

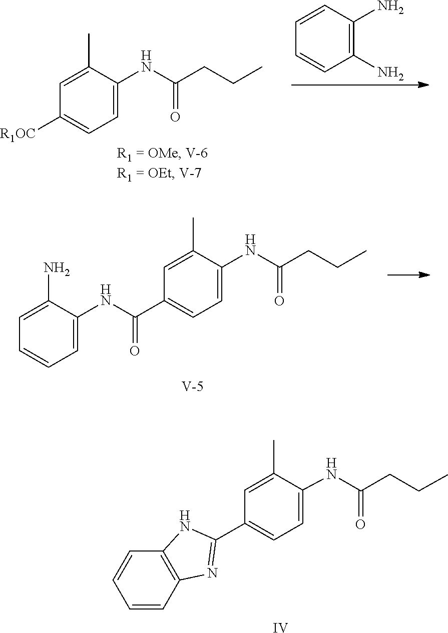

Step (1), reacting methyl 3-methyl-4-n-butyrylaminobenzoate or ethyl 3-methyl-4-n-butyrylaminobenzoate, with o-phenylenediamine in the presence of an acidic reagent or a basic reagent to obtain the compound of formula V-5;

Step (2), reacting the compound of formula V-5 in the presence of the acidic reagent or the basic reagent in the step (1), to obtain the compound of formula IV;

In particular, the step (1) and the step (2) are carried out in one-pot method, that is, in the presence of the acidic reagent or the basic reagent, the compound of formula V-5 prepared in the step (1) continually reacts under the reaction condition of the step (1) without separation to obtain the compound of formula IV;

In particular, in the step (1), the acidic reagent used is one selected from the group consisting of conventional inorganic protonic acids, organic carboxylic is acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;

Particularly in the step (1), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof.

In particular, in the step (2), the acidic reagent or the basic reagent used is the same as defined in the step (1).

Particularly in the step (2), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;

In particular, in the step (1) and the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, is and N-methylpyrrolidone, or a mixture thereof Alternatively, a solvent-free reaction condition is used.

According to one embodiment of the present disclosure, wherein the compound of formula VI-1 is prepared by one of the following methods:

Method IV:

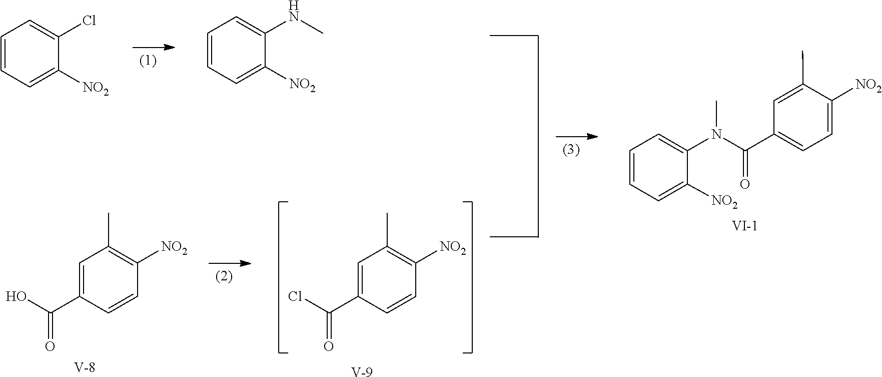

Step (1), reacting o-nitrochlorobenzene in a methylamine aqueous solution under heating conditions to obtain o-nitrobenzylamine;

Step (2), conducting a chlorination reaction of the compound of formula V-8 to obtain the compound of formula V-9;

Step (3), reacting the compound of formula V-9 with o-nitrobenzylamine to obtain the compound of formula VI-1;

Particularly in the step (1), the concentration of the methylamine aqueous solution is 20-30%, and the reaction is carried out at 100-150° C. and 3-10 atm;

In particular, the step (2) and the step (3) are carried out in a solvent;

In particular, the step (2) and the step (3) are carried out in one-pot method, that is, the compound of formula V-9 prepared in the step (2) is directly subjected to the reaction of the step (3) without separation;

wherein, the chlorinating reagent used in the chlorination reaction, the basic reagent and the solvent in the steps (2) and (3) of the reaction process are the same as defined for the chlorinating reagent used in the chlorination reaction, the basic reagent and the solvent in the steps (1) and (2) of the above-mentioned method I;

Method V:

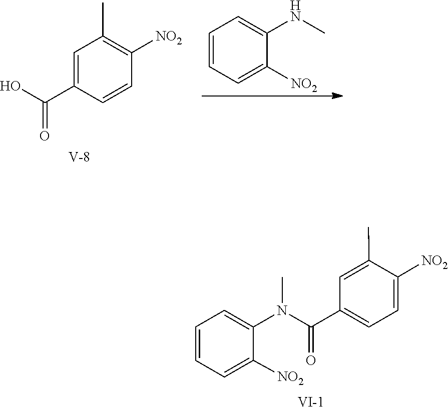

reacting the compound of formula V-8 with o-nitrobenzylamine in the presence of an acidic reagent or a condensation reagent in a solvent to obtain the compound of formula VI-1;

In particular, the acidic reagent, the condensation reagent and the solvent in the reaction process are the same as defined for the acidic reagent, the condensation reagent and the solvent used in the step (1) of the above-mentioned method II;

Method VI:

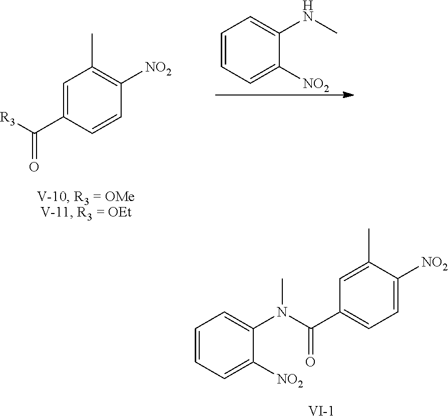

reacting the compound of formula V-10 or the compound of formula V-11 with o-nitrobenzylamine in the presence of an acidic reagent or a basic reagent in a solvent to obtain the compound of formula VI-1;

In particular, the acidic reagent, the basic reagent and the solvent in the reaction process are the same as defined for the acidic reagent, the basic reagent and the solvent in the step (1) of the above-mentioned method III;

Method VII:

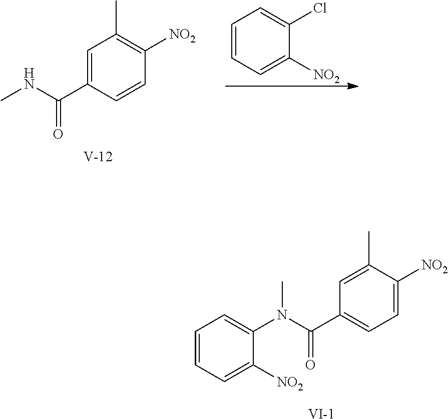

reacting the compound of formula V-12 with o-nitrochlorobenzene in the presence of a basic reagent, a transition metal compound and its complex in a solvent to obtain the compound of formula VI-1;

In particular, the basic reagent, the transition metal compound and its complex, and the solvent in the reaction process are the same as defined for the basic reagent, the transition metal compound and its complex, and the solvent in the above-mentioned method Six;

Method VIII:

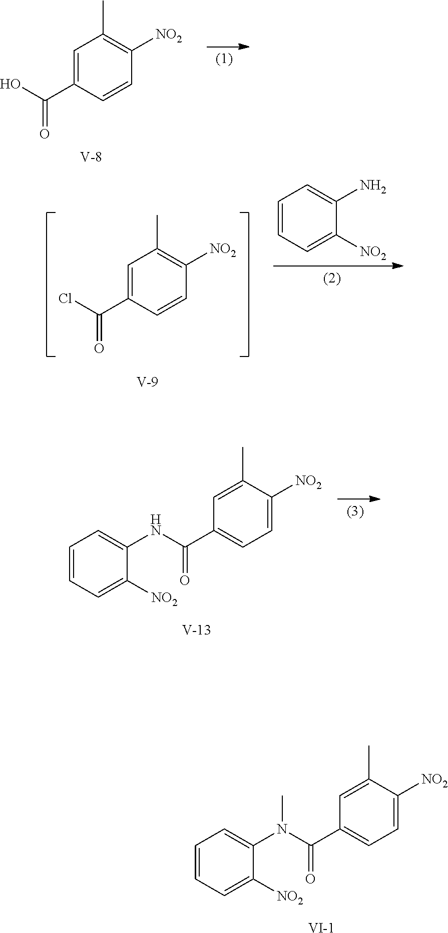

Step (1), preparing 3-methyl-4-nitrobenzoyl chloride through chlorination reaction with 3-methyl-4-nitrobenzoic acid as a starting material;

Step (2), reacting the produced compound of formula V-9 with o-nitrobenzylamine in the presence of a basic reagent to obtain the compound of formula V-13;

Step (3), reacting the compound of formula V-13 with a methylating reagent to prepare the compound of formula VI-1;

In particular, the step (1) and the step (2) are carried out in a solvent;

In particular, the step (1) and the step (2) are carried out in one-pot method, that is, the compound of formula V-9 prepared in the step (1) is directly subjected to the reaction of the step (2) without separation;

wherein, the chlorinating reagent used in the chlorination reaction, the basic reagent and the solvent in the steps (1) and (2) of the reaction process are the same as defined for the chlorinating reagent used in the chlorination reaction, the basic reagent and the solvent in the steps (1) and (2) of the above-mentioned method I;

Particularly in the step (3), the methylating agent is selected from the group consisting of methyl iodide, dimethyl sulfate and dimethyl carbonate;

Particularly in the step (3), the reaction is carried out in the presence of a basic reagent in a solvent,

Particularly in the step (3), the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;

Particularly in the step (3), the solvent is one selected from the group consisting of tetrahydrofuran, dioxane, ethylene glycol dimethyl ether, methanol, ethanol, isopropanol, n-butanol, tert-butanol, ethylene glycol, acetone, acetonitrile, N,N-dimethylformamide, N,N-dimethylacetamide, N-methylpyrrolidone, dimethyl sulfoxide and water; or a mixture thereof;

According to one embodiment of the present disclosure, provided is an intermediate or a salt thereof for preparing the benzimidazole-substituted phenyl-n-butyramide-based compound of formula III, wherein the intermediate is selected from the group consisting of the compounds of formulae IV, V-3, V-4, V-5, and VI-1:

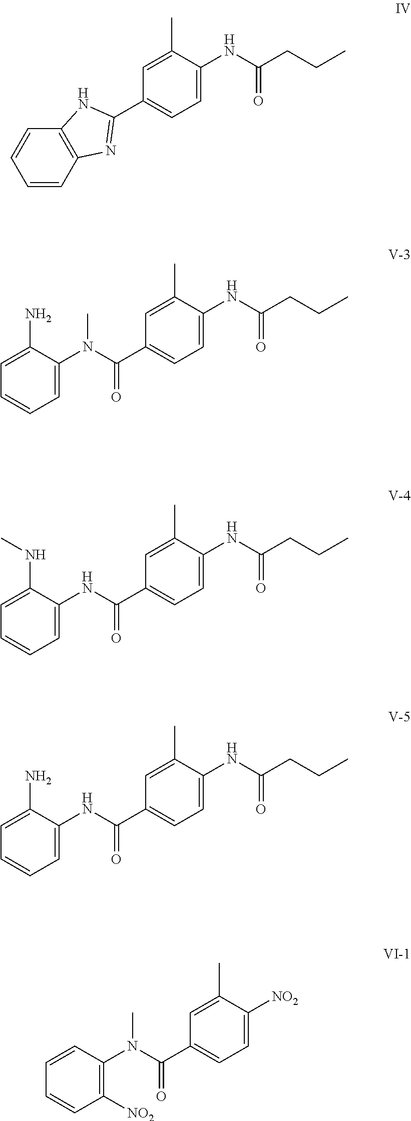 [SMILES: CCCC(=O)Nc1ccc(-c2nc3ccccc3[nH]2)cc1C ; CCCC(=O)Nc1ccc(C(=O)N(C)c2ccccc2N)cc1C ; CCCC(=O)Nc1ccc(C(=O)Nc2ccccc2NC)cc1C ; CCCC(=O)Nc1ccc(C(=O)Nc2ccccc2N)cc1C ; Cc1cc(C(=O)N(C)c2ccccc2[N+](=O)[O-])ccc1[N+](=O)[O-]]

According to an embodiment of the present disclosure, provided is a method for preparing the compound of formula II, comprising:

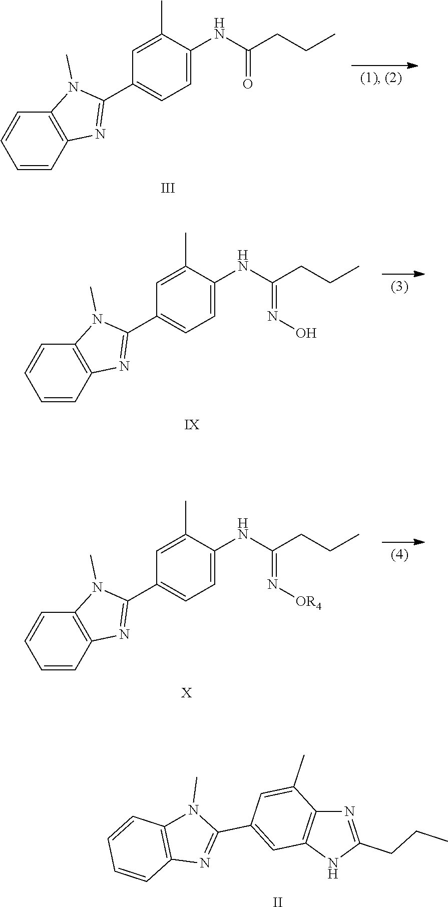

Step (1), reacting the compound of formula III with a chlorinating reagent,

Step (2), reacting the chlorination reaction mixture obtained in the step (1) with a hydroxylamine reagent to obtain the compound of formula IX;

Step (3), reacting the compound of formula IX with an acid chloride or acid anhydride reagent to obtain a compound of formula X;

Step (4), reacting the compound of formula X in the presence of a basic reagent to obtain the compound of formula II,

Particularly in the step (1), the chlorinating reagent used is one selected from the group consisting of thionyl chloride, phosphorus oxychloride, phosphorus pentachloride, oxalyl chloride, phosgene, and bis(trichloromethyl) carbonate (triphosgene), or a mixture thereof; the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, and methyl tert-butyl ether, or a mixture thereof,

Particularly in the step (2), the hydroxylamine reagent used is selected from the group consisting of a free base-type hydroxylamine, hydroxylamine hydrochloride, or a salt formed by hydroxylamine,

Particularly in the step (3), in the compound of formula X, R₄ is a carboxylic acid acyl of —C(═O)R₅, a sulfonyl of —SO₂R₆, an alkoxycarbonyl of —C(═O)—OR₇, an alkylaminocarbonyl of —C(═O)—NR₈R₉, or an alkoxyphosphoryl of —P(═O)(OR₁₀)₂;

wherein, R₅ to R₁₀ are each independently hydrogen, substituted or unsubstituted C₁-C₂₀ linear or branched or cyclic alkyl, substituted or unsubstituted C₁-C₂₀ linear or branched or cyclic alkenyl, substituted or unsubstituted benzyl, substituted or unsubstituted C₆-C₂₀ aryl; in the case that R₅ to R₁₀ are substituted C₁-C₂₀ linear or branched or cyclic alkyl, substituted C₁-C₂₀ linear or branched or cyclic alkenyl, substituted benzyl, or substituted C₆-C₂₀ aryl, the substituent is selected from the group consisting of cyano, nitro, amino, hydroxyl, mercapto, halogen, phenyl, C₁-C₂₀ linear or branched or cyclic alkyl, C₁-C₂₀ linear or branched or cyclic alkenyl, and C₁-C₂₀ linear or branched or cyclic alkoxy,

Particularly in the step (3), the acid chloride or acid anhydride reagent used is an acid chloride or acid anhydride corresponding to R₄, preferably is one selected from the group consisting of acetyl chloride, trifluoroacetyl chloride, benzoyl chloride, p-nitrobenzoyl chloride, p-chlorobenzoyl chloride, methanesulfonyl chloride, trifluoromethanesulfonyl chloride, p-toluenesulfonyl chloride, acetic anhydride, trifluoroacetic anhydride, benzoic anhydride, p-nitrobenzoic anhydride, p-chlorobenzoic anhydride, methanesulfonic anhydride, trifluoromethanesulfonic anhydride, p-toluenesulfonic anhydride, methyl chloroformate, ethyl chloroformate, benzyl chloroformate, di-tert-butyl dicarbonate, N,N-dimethylchloroformamide, diethoxyphosphoryl chloride, and so on, or a mixture thereof,

Particularly in the step (3), the reaction is carried out in the presence of a basic reagent, the basic reagent is one selected from the group consisting of lithium carbonate, lithium hydroxide, lithium tert-butoxide, sodium carbonate, sodium bicarbonate, sodium hydroxide, sodium phosphate, sodium methoxide, sodium ethoxide, sodium isopropoxide, sodium tert-butoxide, potassium carbonate, potassium bicarbonate, potassium hydroxide, potassium phosphate, potassium methoxide, potassium ethoxide, potassium tert-butoxide, cesium carbonate, cesium hydroxide, magnesium carbonate, magnesium hydroxide, magnesium phosphate, magnesium oxide, magnesium methoxide, magnesium ethoxide, magnesium isopropoxide, magnesium tert-butoxide, triethylamine, diisopropylamine, diisopropylethylamine, tri-n-butylamine, pyridine, 2-methylpyridine, 2,6-dimethylpyridine, 4-dimethylaminopyridine, tetrahydropyrrole, morpholine, piperidine, and 2,2,6,6-tetramethylpiperidine; or a mixture thereof,

Particularly in the step (3), the reaction is carried out in a solvent, the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, methyl tert-butyl ether and water, or a mixture thereof,

Particularly in the step (4), the basic reagent used is one selected from the group consisting of lithium carbonate, lithium hydroxide, lithium tert-butoxide, sodium carbonate, sodium bicarbonate, sodium hydroxide, sodium phosphate, sodium methoxide, sodium ethoxide, sodium isopropoxide, sodium tert-butoxide, potassium carbonate, potassium bicarbonate, potassium hydroxide, potassium phosphate, potassium methoxide, potassium ethoxide, potassium tert-butoxide, cesium carbonate, cesium hydroxide, magnesium carbonate, magnesium hydroxide, magnesium phosphate, magnesium oxide, magnesium methoxide, magnesium ethoxide, magnesium isopropoxide, magnesium tert-butoxide, triethylamine, diisopropylamine, diisopropylethylamine, tri-n-butylamine, pyridine, 2-methyl pyridine, 2,6-demethylpyridine, 4-dimethylaminopyridine, tetrahydropyrrole, morpholine, piperidine, and 2,2,6,6-tetramethylpiperidine, or a mixture thereof,

Particularly in the step (4), the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, methyl tert-butyl ether, and water, or a mixture thereof.

According to one embodiment of the present disclosure, the step (3) and the step (4) are performed in one-pot method.

According to one embodiment of the present disclosure, the step (1) to the step (4) are performed in one-pot method.

According to one embodiment of the present disclosure, provided is an intermediate or a salt thereof for preparing the compound of formula II, wherein the intermediate is selected from the group consisting of the compounds of formulae IX and X, or salts thereof:

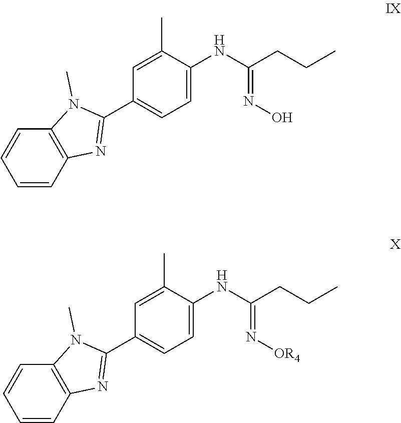

wherein, R₄ in the compound of formula X is defined as —C(═O)R₅, —SO₂R₆, —C(═O)—OR₇, —C(═O)—NR₈R₉, —P(═O)(OR₁₀)₂, wherein, R₅ to R₁₀ are each independently hydrogen, substituted or unsubstituted C₁-C₂₀ linear or branched or cyclic alkyl, substituted or unsubstituted C₁-C₂₀ linear or branched or cyclic alkenyl, substituted or unsubstituted benzyl, substituted or unsubstituted C₆-C₂₀ aryl; in the case that R₅ to R₁₀ are substituted C₁-C₂₀ linear or branched or cyclic alkyl, substituted C₁-C₂₀ linear or branched or cyclic alkenyl, substituted benzyl, or substituted C₆-C₂₀ aryl, the substituent is selected from the group consisting of cyano, nitro, amino, hydroxyl, mercapto, halogen, phenyl, C₁-C₂₀ linear or branched or cyclic alkyl, C₁-C₂₀ linear or branched or cyclic alkenyl, and C₁-C₂₀ linear or branched or cyclic alkoxy.

Beneficial Effects

The invention provides various preparation methods and uses of benzimidazole-substituted phenyl-n-butyramide, and one of its uses is to prepare the bisbenzimidazole intermediate of the antihypertension drug Telmisartan. The method avoids nitration reaction and polyphosphoric acid cyclization reaction, and avoids the generation of a large amount of waste acid reaction solution from the source. The synthetic method embodied in the invention has the advantages of simplicity and high efficiency, mild conditions and less pollutants, etc., and is suitable to be developed as a green and sustainable production process.

### DETAILED EMBODIMENTS

In order to understand the features and effects of the present invention for those with ordinary knowledge in the art, general descriptions and definitions of terms and words mentioned in the specification and the claims of the patent application are provided below. Unless otherwise specified, all technical and scientific terms used herein have the ordinary meanings understood by those skilled in the art to the present invention, and in case of conflict, the definitions in this specification shall prevail.

As used herein, the terms “comprise”, “include”, “have”, “contain” or any other similar terms are open-ended transitional phrases, which intended to cover non-exclusive inclusions. For example, a composition or article containing a plurality of elements is not limited to only the elements listed herein, but can also include other elements not expressly listed, but which are generally inherent to the composition or article. In addition, unless expressly stated to the contrary, the term “or” refers to an inclusive “or” rather than an exclusive “or”. For example, the condition “A or B” is satisfied by any of the followings: A is true (or present) and B is false (or absent), A is false (or absent) and B is true (or present), both A and B are true (or present). In addition, the terms “comprise”, “include”, “have”, “contain” herein intend to be interpreted as having specifically disclosed and encompassing both “consisting of” and “substantially consisting of” and other closed or semi-closed transitional phrase.

All features or conditions herein defined as numerical ranges or percentage ranges are for brevity and convenience only. Accordingly, the description of numerical ranges or percentage ranges should be considered to encompass and specifically disclose all possible sub-ranges and individual numerical values within the range, particularly integer numerical values. For example, a range description of “1 to 8” should be deemed to have specifically disclosed all subranges, such as 1 to 7, 2 to 8, 2 to 6, 3 to 6, 4 to 8, 3 to 8, etc., particularly are sub-ranges bounded by all integer values, and should be deemed to have disclosed the individual values within the specifically disclosed range, such as 1, 2, 3, 4, 5, 6, 7, 8, etc. Unless otherwise indicated, the foregoing method of interpretation applies to all content throughout this disclosure, whether broad or not.

If a quantity or other value or parameter is expressed as a range, a preferred range, or a series of upper and lower limits, it should be understood that all the ranges constituted by any upper limit of the range or preferred value and the lower limit of the range or preferred value have been specifically disclosed herein, whether or not those ranges are disclosed separately. Furthermore, unless otherwise indicated, when a numerical range is referred to herein, the range shall include its endpoints and all integers and fractions within the range.

As used herein, numerical values should be understood to have an accuracy of significant digits of the numerical value, provided that the purpose of the invention can be achieved. For example, the number 40.0 should be understood to cover the range from 39.50 to 40.49.

Where Markush group or alternative terminology is used herein to describe features or examples of the invention, those skilled in the art will understand that all subgroups of elements or any individual element within the Markush group or list of options may also be used to describe the invention. For example, if X is described as “selected from the group consisting of X₁, X₂ and X₃”, it also means that the claim that X is X₁ and that X is X₁ and/or X₂ have been fully described. Further for the case that Markush group or alternative terminology is used herein to describe features or examples of the invention, those skilled in is the art will understand that all subgroups of elements or any individual element within the Markush group or list of options may also be used to describe the invention. Accordingly, for example, if X is described as “selected from the group consisting of X₁, X₂ and X₃” and Y is described as “selected from the group consisting of Y₁, Y₂ and Y₃” means that the claim that X is X₁ or X₂ or X₃ and Y is Y₁ or Y₂ or Y₃ has been fully described.

The following detailed embodiments are merely exemplary in nature and are not intended to limit the invention and its uses. Furthermore, the present applicant is not intented to be bound by any theory presented in the preceding Background Art or Summary of the Invention or in the following detailed embodiments or examples.

Embodiments of the invention are illustrated by the following examples. It is to be understood, however, that embodiments of the present invention are not limited to the specific details in the following examples, because other variations known and apparent to those of ordinary skill in the art in view of the present disclosure are considered to be included in the present invention.

### EXAMPLE 1

Method of preparing the intermediate IV through the intermediates V-2 and V-5 from 3-methyl-4-n-butyrylaminobenzoic acid (V-1).

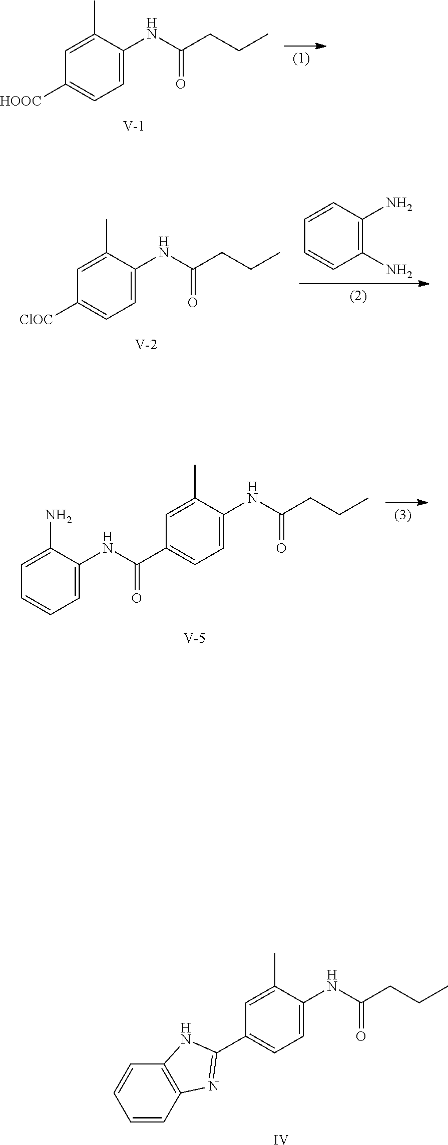

1) Preparation of the intermediate V-5

3-methyl-4-n-butyrylaminobenzoic acid (22.1 g, 100 mmol), triphosgene (10.4 g, 35 mmol, 0.35 eq) and tetrahydrofuran (70 mL) were added to a three-necked flask, heated to 40 to 45° C. and stirred well. Then N,N-dimethylformamide (365 mg, 5 mmol, 0.05 eq) was added, heated and stirred at 40-45° C. for 5-6 hours, cooled and then transferred to a constant pressure funnel for use. To another three-necked flask, were added o-phenylenediamine (11.9 g, 110 mmol, 1.1 eq), acetonitrile (95 mL) and aqueous sodium hydroxide solution (8 g, 200 mmol, 2 eq dissolved in 200 mL of water), stirred well under an ice bath at 0 to 5° C. The prepared acid chloride was slowly added dropwise from the constant pressure funnel to the reaction under stirring in an ice bath at 0 to 5° C. After the dropwise addition was completed, the resultant was heated to room temperature and stirred for 1 hour. is The reaction was heated and concentrated to remove about half the volume of the organic solvent, then water (100 mL) was added and stirred well. The precipitated solid was collected and fully dried to obtain 28.1 g of a pale yellow solid with a yield of 85%.

Characterization data of V-5:

¹H NMR (d₆-DMSO 400 MHz) δ: 0.96 (t, J=8 Hz, 3H), 1.61-1.65 (m, 2H), 2.30 (s, 3H), 2.38-2.39 (m, 2H), 4.89 (s, 2H), 6.59-6.62 (m, 1H), 6.79 (d, J=8 Hz, 1H), 6.97 (t, J=8 Hz, 1H), 7.18 (d, J=8 Hz, 1H), 7.63 (d, J=8 Hz, 1H), 7.79-7.81 (m, 1H), 7.86 (s, 1H), 9.37 (s, 1H), 9.61 (s, 1H).

LR-MS (ESI) m/z: 312 (M+H)⁺.

2) Preparation of the intermediate IV

The intermediate V-5 (2.0 g, 6.5 mmol) and p-toluenesulfonic acid monohydrate (120 mg, 0.6 mmol, 0.1 eq) were added to toluene (20 mL) and stirred well. The reaction was heated under reflux with water separation overnight. After the reaction was cooled, solids were precipitated. Most of the solvent was removed by concentration, and then the resultant was added with sodium hydroxide aqueous solution (concentration about 10%) to adjust the pH to 11 to 12 and stirred well. The precipitated solid was collected and fully dried to obtain 1.5 g of an off-white solid with a yield of 80%.

Characterization data of IV:

¹H NMR (d₆-DMSO 400 MHz) δ: 0.94 (t, J=8 Hz, 3H), 1.62-1.70 (m, 2H), 2.31 (s, 3H), 2.37 (t, J=8 Hz, 2H), 7.17-7.19 (m, 2H), 7.57-7.80 (m, 4H), 7.95 (d, J=8 Hz, 1H), 8.04 (s, 1H), 9.32 (s, 1H).

LR-MS (ESI) m/z: 294 (M+H)⁺.

### EXAMPLE 2

Method of preparing the intermediate VI-1 from the intermediate V-8.

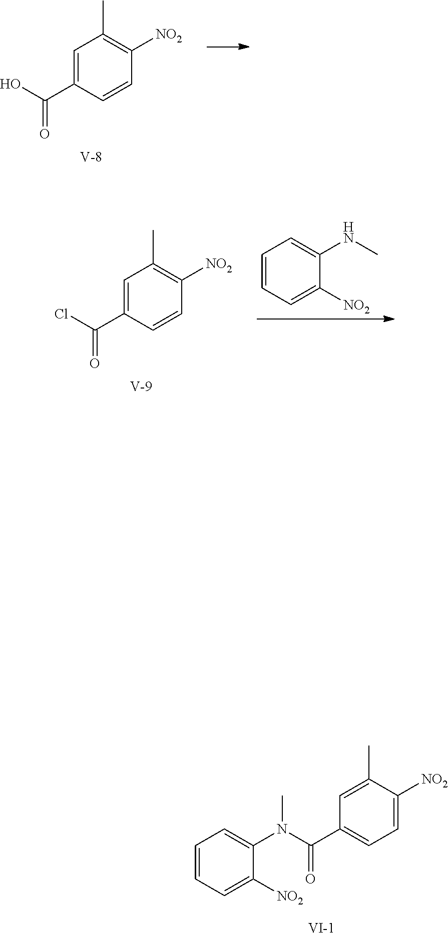

V-8 (10 g, 55.2 mmol) was insoluble in 50 ml of dry acetonitrile, added with DMF (400 mg, 5.5 mmol), triphosgene (6.5 g, 22 mmol), reflux with acetonitrile for 2 h under N₂ protection, and the solution became clear. TLC showed that the reaction of the starting materials was complete. In another round-bottomed flask, o-nitrobenzylamine (8.4 g, 55.2 mmol) was dissolved in 50 ml of dry acetonitrile, added with potassium carbonate (22.9 g, 165.6 mmol). The acid chloride reaction solution was added dropwise to the o-nitrobenzylamine under an ice bath, the resultant was refluxed with acetonitrile for 2 h under N₂ protection. The reaction solution was concentrated, and the residue was added with 150 ml of water, extracted with 150 ml of EA. The organic phase was washed with saturated sodium chloride, dried, rotary evaporated, and subjected to column chromatography to obtain 14.5 g of VI-1 as a pale yellow solid with a yield of 83.3%.

Characterization data of VI-1:

¹H NMR (400 MHz, MeOD): 7.92 (dd, J=8.3, 1.5 Hz, 1H), 7.64-7.76 (m, 3H), 7.48-7.55 (m, 1H), 7.32 (d, J=1.9 Hz, 1H), 7.24 (dd, J=8.3, 1.9 Hz, 1H), 3.47 (s, 3H), 2.41 (s, 3H).

LR-MS (ESI) m/z: 316.6 (M+H)⁺.

### EXAMPLE 3

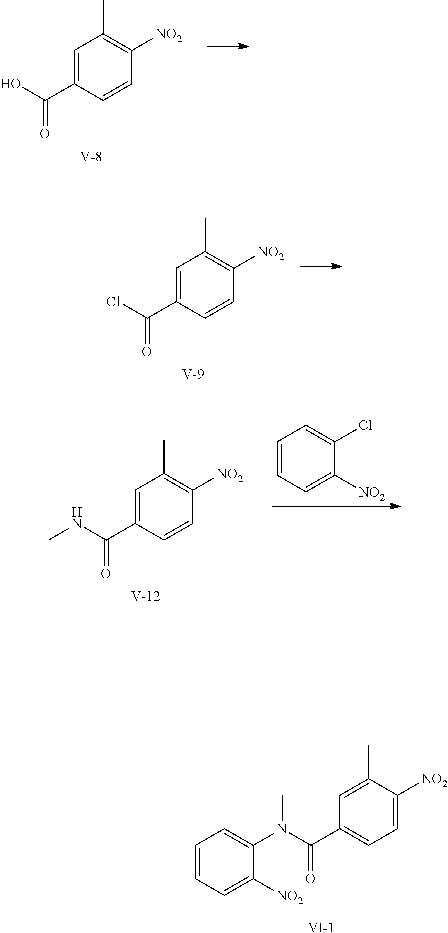

1) Preparation of V-12:

V-8 (5 g, 27.6 mmol) was added with 25 ml of acetonitrile, followed by addition of DMF (200 mg, 2.76 mmol), triphosgene (3.27 g, 11.0 mmol), and refluxed with acetonitrile under N₂ protection for 2 h. To another round bottom flask, 30 ml of methylamine alcohol solution was added, and an acid chloride solution was added dropwise under an ice bath, followed by addition of 100 ml of EA. The organic phase was washed twice with saturated brine, dried and rotary evaporated to obtain 5 g of V-12 as a yellow solid with a yield of 93.3%. Characterization data of V-12:

¹H NMR (400 MHz, CDCl₃): δ 7.94 (dd, J=8.4, 1.7 Hz, 1H), 7.75 (d, J=1.9 Hz, 1H), 7.67 (dd, J=8.4, 1.9 Hz, 1H), 6.62 (s, 1H), 3.00 (d, J=4.8Hz, 3H), 2.59 (s, 3H).

LR-MS (ESI) m/z: 195.4 (M+H)⁺.

2) Preparation of VI-1

V-12 (388 mg, 2 mmol), o-nitrochlorobenzene (347 mg, 2.2 mmol), Pd(dppf)Cl₂ (73 mg, 0.1 mmol), Cs₂CO₃ (1.95 g, 6 mmol) were added with 10 ml of toluene, heated at an external temperature of 110° C. under N₂ protection is for 10 h, and filtered. The filtrate was diluted with 20 ml of EA, and the organic phase was washed with saturated brine, dried, rotary evaporated, and subjected to column chromatography to obtain 530 mg of VI-1 as a pale yellow solid with a yield of 84.0%.

Characterization data of VI-1:

¹H NMR (400 MHz, MeOD): 7.92 (dd, J=8.3, 1.5 Hz, 1H), 7.64-7.76 (m, 3H), 7.48-7.55 (m, 1H), 7.32 (d, J=1.9 Hz, 1H), 7.24 (dd, J=8.3, 1.9 Hz, 1H), 3.47 (s, 3H), 2.41 (s, 3H).

LR-MS (ESI) m/z: 316.6 (M+H)⁺.

### EXAMPLE 4

Method of preparing the intermediate III from the intermediate IV.

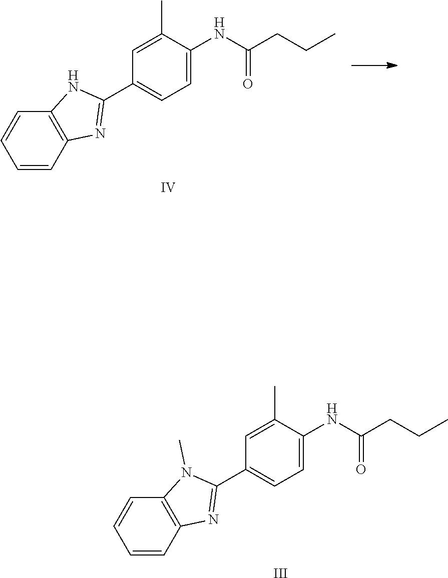 [SMILES: CCCC(=O)Nc1ccc(-c2nc3ccccc3[nH]2)cc1C ; "IV" ; CCCC(=O)Nc1ccc(-c2nc3ccccc3n2C)cc1C ; "III"]

IV (586 mg, 2 mmol), dimethyl sulfate (277 mg, 2.2 mmol, 1.1 eq), potassium carbonate (414 mg, 3.0 mmol, 1.5 eq) were added with acetone (10 mL) at room temperature and stirred well. The reaction was heated to reflux for 2 hours, concentrated to remove most of the solvent, and then separated and purified by column chromatography to obtain 567 mg of an off-white solid with a yield of 92%.

Characterization data of III:

¹H NMR (d₆-DMSO 400 MHz) δ: 1.02 (t, J=8 Hz, 3H), 1.76-1.83 (m, 2H), 2.28 (s, 3H), 2.44 (t, J=8 Hz, 2H), 3.83 (s, 3H), 7.31-7.34 (m, 2H), 7.38-7.40 (m, 1H), 7.45 (d, J=8 Hz, 1H), 7.54 (s, 1H), 7.80-7.93 (m, 1H). LR-MS (ESI) m/z: 308 (M+H)⁺.

### EXAMPLE 5

Method of preparing the intermediate IV from V-1.

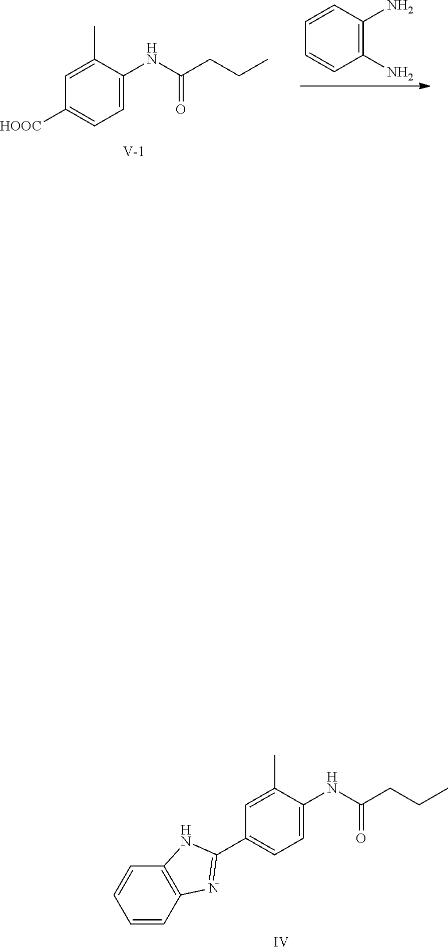

V-1 (4.4 g, 20 mmol), p-toluenesulfonic acid monohydrate (380 mg, 2 mmol, 0.1 eq), and o-phenylenediamine (2.2 g, 21 mmol, 1.05 eq) were added to a three-necked flask, added with toluene (40 mL) and stirred well. The reaction was heated under reflux with water separation overnight. After the reaction was cooled, solids were precipitated. Most of the solvent was removed by concentration, and then the resultant was added with sodium hydroxide aqueous solution (concentration about 10%) to adjust pH to 11 to 12 and stirred well. The precipitated solid was collected and fully dried to obtain 4.4 g of an off-white solid with a yield of 75%.

Characterization data of IV:

¹H NMR (d₆-DMSO 400 MHz) δ: 0.94 (t, J=8 Hz, 3H), 1.62-1.70 (m, 2H), 2.31 (s, 3H), 2.37 (t, J=8 Hz, 2H), 7.17-7.19 (m, 2H), 7.57-7.80 (m, 4H), 7.95 (d, J=8 Hz, 1H), 8.04 (s, 1H), 9.32 (s, 1H).

LR-MS (ESI) m/z: 294 (M+H)⁺.

It is the same as characterization data of the product IV obtained in Example 1.

### EXAMPLE 6

Method of preparing the intermediate III from V-6.

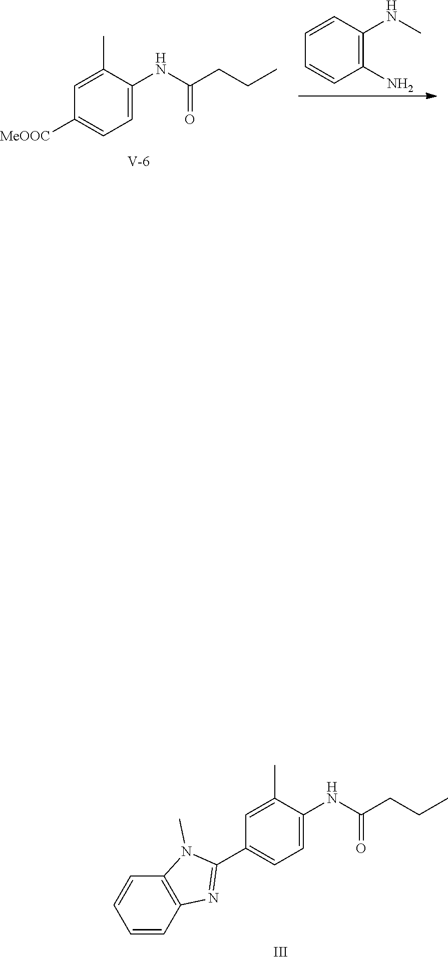

V-6 (4.7 g, 20 mmol), p-toluenesulfonic acid monohydrate (380 mg, 2 mmol, 0.1 eq), and N-methyl-o-phenylenediamine (2.6 g, 21 mmol, 1.05 eq) were added to a three-necked flask, added with toluene (40 mL) and stirred well. The reaction was heated under reflux with water separation for 30 hours. After the reaction was cooled, solids were precipitated. Most of the solvent was removed by concentration, and then the resultant was added with sodium hydroxide aqueous solution (concentration about 10%) to adjust pH to 11 to 12 and stirred well. The precipitated solid was collected and fully dried to obtain 3.4 g of an off-white solid with a yield of 55%.

Characterization data of III:

¹H NMR (d₆-DMSO 400 MHz) δ: 1.02 (t, J=8 Hz, 3H), 1.76-1.83 (m, 2H), 2.28 (s, 3H), 2.44 (t, J=8 Hz, 2H), 3.83 (s, 3H), 7.31-7.34 (m, 2H), 7.38-7.40 (m, 1H), 7.45 (d, J=8 Hz, 1H), 7.54 (s, 1H), 7.80-7.93 (m, 1H).

LR-MS (ESI) m/z: 308 (M+H)⁺.

The Characterization data are the same as those of the product III obtained in Example 2.

### EXAMPLE 7

Method of preparing the intermediate III from VI-1.

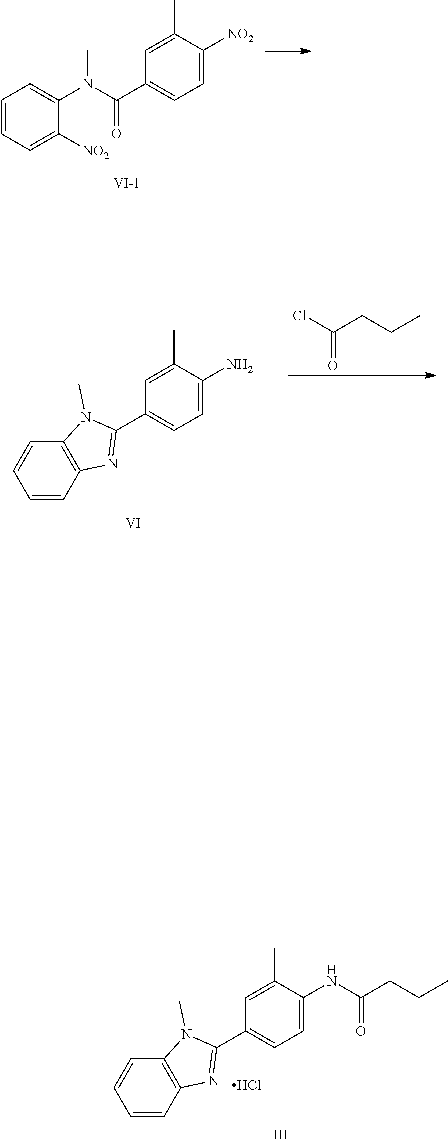

1) Preparation of VI:

VI-1 (10 g, 31 7 mmol) was added to 100 ml of ethanol, added with acetic acid (7.7 g, 128 8 mmol), 1 g of 5% Pd/C, heated at an external temperature of 70° C. under the hydrogen pressure (10 kg of hydrogen pressure). After 10 hours, the reaction solution was filtered, and the filtrate was concentrated to remove most of the solvent, added with saturated NaOH to adjust pH to 10, added with 200 ml of DCM for extraction. The organic phase was washed with saturated brine, dried, rotary evaporated, and slurried with a small amount of ethanol to obtain 6.5 g of a pale yellow solid with a yield of 86.4%. ¹H NMR (500 MHz, DMSO-d6): δ 7.59 (d, J=7.2 Hz, 1H), 7.49-7.55 (m, 1H), 7.46 (d, J=2.1 Hz, 1H), 7.39-7.44 (m, 1H), 7.15-7.26 (m, 2H), 6.75 (d, J=8.2 Hz, 1H), 5.37 (s, 2H), 3.83 (s, 3H), 2.15 (s, 3H). LR-MS (ESI) m/z: 238.2 (M+H)⁺.

2) Preparation of III

In a three-necked flask, VI (237 mg, 1.0 mmol) was added to acetonitrile (5 mL) and aqueous sodium hydroxide solution (80 mg, 2.0 mmol, 2.0 eq, dissolved in 5 mL of water), and the resultant was stirred well under an ice bath at 0 to 5° C. A solution of n-butyryl chloride (106 mg, 1.0 mmol, 1.0 eq) in acetonitrile (3 mL) was slowly added dropwise to the reaction under stirring in an ice bath at 0 to 5° C. After the dropwise addition was completed, the resultant was heated to room temperature and stirred for half an hour. The reaction was added with water (20 mL) and stirred well. The precipitated solid was collected, dissolved by adding a small amount of isopropanol, added with concentrated hydrochloric acid under stirring to precipitate a white precipitate. The solid was collected and fully dried to obtain 327 mg of a white solid with a yield of 95%.

Characterization data of III (hydrochloride):

¹H NMR (d₆-DMSO 400 MHz) δ: 0.96 (t, J=8 Hz, 3H), 1.63-1.67 (m, 2H), 2.38 (s, 3H), 2.44 (t, J=8 Hz, 2H), 4.06 (s, 3H), 7.61-7.66 (m, 2H), 7.79-7.92 (m, 4H), 8.03-8.05 (m, 1H), 9.66 (s, 1H).

LR-MS (ESI) m/z: 308 (M+H)⁺.

### EXAMPLE 8

Method of preparing II from III.

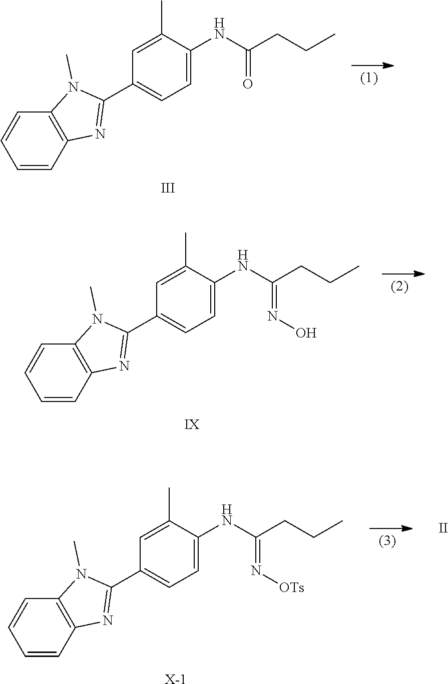

1) Preparation of IX:

Intermediate III (hydrochloride) (6.88 g, 20 mmol), triphosgene (2.38 g, 8 mmol, 0.4 eq) and acetonitrile (30 mL) were heated to 80 to 85° C. and stirred well. Then N,N-dimethylformamide (73 mg, 1.0 mmol, 0.05 eq) was added thereto, and the resultant was maintained at 80 to 85° C. under heating and stirring for 2 hours. After cooled to room temperature, the reaction was added to a solution of hydroxylamine (990 mg, 30 mmol, 1.5 eq; acetonitrile 10 mL) in acetonitrile with stirring, and kept stirring at room temperature for 1 hour. After the reaction was complete, the reaction was added with sodium hydroxide aqueous solution (10%) to adjust pH to 10 to 11, cooled to 0 to 5° C. to precipitate an off-white precipitate. The solid was collected and fully dried to obtain 5.9 g of an off-white solid with a yield of 92%.

Characterization data of IX:

¹H NMR (CDCl₃, 400 MHz) δ: 0.88 (t, J=8 Hz, 3H), 1.42-1.46 (m, 2H), 2.32 (t, J=8 Hz, 2H), 2.39 (s, 3H), 3.91 (s, 3H), 7.02 (brs, 1H), 7.24 (d, J=8 Hz, 1H), 7.32-7.34 (m, 2H), 7.40-7.42 (m, 1H), 7.58 (d, J=8 Hz, 1H), 7.72 (s, 1H), 7.82-7.85 (m, 1H).

LR-MS (ESI) m/z: 323 (M+H)⁺.

2) Preparation of X-1:

IX (6.4 g, 20 mmol) was dissolved in acetonitrile (20 mL). Then triethylamine (2.42 g, 24 mmol, 1.2 eq) was added to the reaction, and then p-toluenesulfonyl chloride (4.2 g, 22 mmol, 1.1 eq) was slowly added to the reaction at 0 to 5° C. After the addition was complete, the resultant was raised to room temperature to react for 1-2 hours. Ethyl acetate (10 mL) was added for extraction, and the aqueous phase was removed by separation. The organic phase was washed once with an aqueous sodium hydroxide solution (10%, 10 mL), and concentrated to precipitate a solid. The precipitated solid was collected and fully dried to obtain the product as a pale yellow solid.

3) Preparation of II:

X-1 (9.5 g, 20 mmol) was dissolved in acetonitrile (20 mL). Then an aqueous sodium hydroxide solution (3.2 g, 80 mmol, 4.0 eq, 20 mL of water) was added to the reaction, and after the addition was completed, the reaction was raised to room temperature for 3 to 4 hours. Ethyl acetate (10 mL) was added for extraction, and the aqueous phase was removed by separation. The organic phase was washed once with an aqueous sodium hydroxide solution (10%, 10 mL), and concentrated to precipitate a solid. The precipitated solid was collected and fully dried to obtain 5.5 g of a pale yellow solid with a yield of 90%.

Characterization data of II:

¹H NMR (400 MHz, CDCl₃) δ: 0.79 (t, J=8 Hz, 3H), 1.68 (m, 2H), 2.47 (s, 3H), 2.70 (t, J=8 Hz, 2H), 3.85 (s, 3H), 7.26 (s, 1H), 7.30-7.42 (m, 3H), 7.68 (s, 1H), 7.74-7.77 (m, 1H).

LR-MS (ESI) m/z: 305 (M+H)⁺.

### EXAMPLE 9

Method of preparing II by a “one-pot” sequential reaction from III.

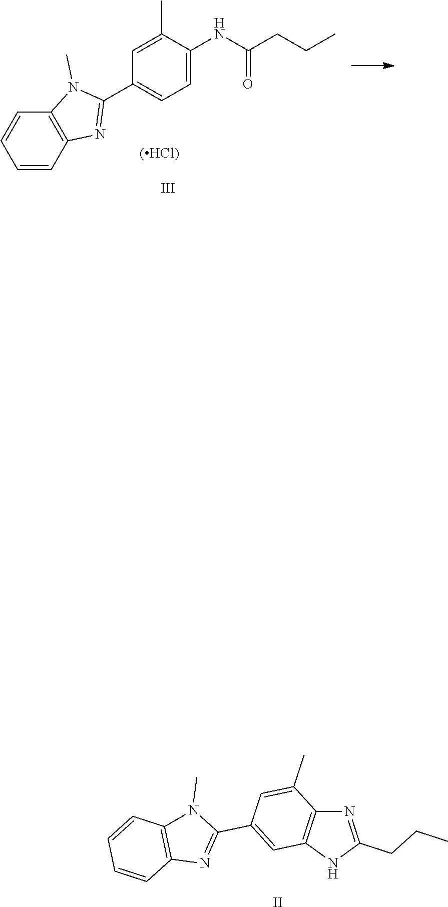

Intermediate III (hydrochloride) (6.88 g, 20 mmol), triphosgene (2.38 g, 8 mmol, 0.4 eq) and acetonitrile (30 mL) were heated to 80 to 85° C. and stirred well. Then N,N-dimethylformamide (73 mg, 1.0 mmol, 0.05 eq) was added thereto, and the resultant was maintained at 80 to 85° C. under heating and stirring for 2 hours. After cooled to room temperature, the reaction was added with hydroxylamine (1.0 g, 30 mmol, 1.5 eq) with stirring, and kept stirring at room temperature for 1 hour. Then triethylamine (8.8 g, 80 mmol, 4.0 eq) was added to the reaction, and then p-toluenesulfonyl chloride (4.2 g, 22 mmol, 1.1 eq) was slowly added to the reaction at 0 to 5° C. After the addition was complete, the resultant was raised to room temperature to react for 3-4 hours. Ethyl acetate (10 mL) was added for extraction, and the aqueous phase was removed by separation. The organic phase was washed once with an aqueous sodium hydroxide solution (10%, 10 mL), and concentrated to precipitate a solid. The precipitated solid was collected and fully dried to obtain 5.2 g of a pale yellow solid with a yield of 85%.

Characterization data of II:

¹H NMR (400 MHz, CDCl₃) δ: 0.79 (t, J=8 Hz, 3H), 1.68 (m, 2H), 2.47 (s, 3H), 2.70 (t, J=8 Hz, 2H), 3.85 (s, 3H), 7.26 (s, 1H), 7.30-7.42 (m, 3H), 7.68 (s, 1H), 7.74-7.77 (m, 1H).

LR-MS (ESI) m/z: 305 (M+H)⁺.

EXAMPLE 10

Method of preparing II by a “one-pot” sequential reaction from IX.

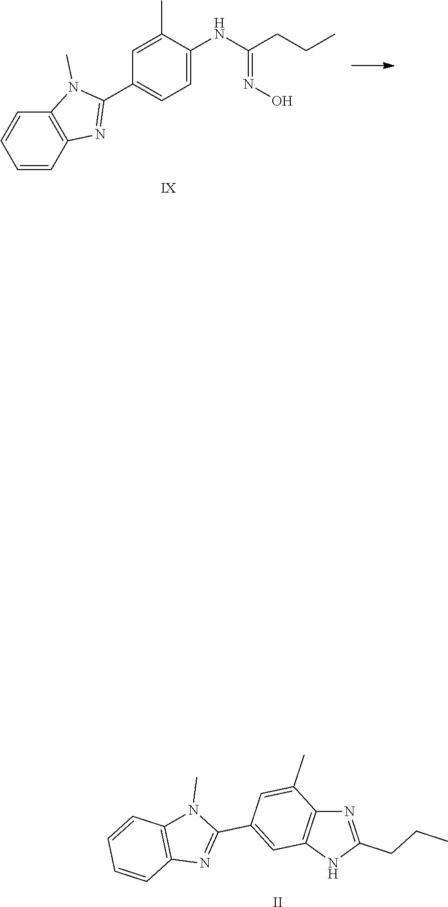

IX (6.4 g, 20 mmol) was dissolved in acetonitrile (20 mL). Aqueous sodium hydroxide solution (3.2 g, 80 mmol, 4.0 eq, 20 mL of water) was added to the reaction, and then p-toluenesulfonyl chloride (4.2 g, 22 mmol, 1.1 eq) was slowly added to the reaction at 0 to 5° C. After the addition was complete, the resultant was raised to room temperature to react for 3 to 4 hours. Ethyl acetate (10 mL) was added for extraction, and the aqueous phase was removed by separation. The organic phase was washed once with an aqueous sodium hydroxide solution (10%, 10 mL), and concentrated to precipitate a solid. The precipitated solid was collected and fully dried to obtain 5.4 g of the product as a pale yellow solid with a yield of 88%.

Characterization data of II:

¹H NMR (400 MHz, CDCl₃) δ: 0.79 (t, J=8 Hz, 3H), 1.68 (m, 2H), 2.47 (s, 3H), 2.70 (t, J=8 Hz, 2H), 3.85 (s, 3H), 7.26 (s, 1H), 7.30-7.42 (m, 3H), 7.68 (s, 1H), 7.74-7.77 (m, 1H).

LR-MS (ESI) m/z: 305 (M+H)⁺.

1. The compound of formula III or a salt thereof: 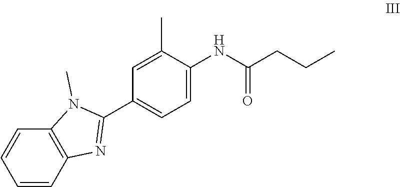 [SMILES: CCCC(=O)Nc1ccc(-c2nc3ccccc3n2C)cc1C]
In particular, the salt is III·HX, a salt form by the compound of formula III with HX which is an acid selected from the group consisting of hydrochloric acid, hydrobromic acid, sulfuric acid, phosphoric acid, formic acid and acetic acid.

2. A method for preparing the compound of formula III, wherein the method is one of the following methods:
Method One: prepared by reacting the compound of formula IV with a methylating reagent, 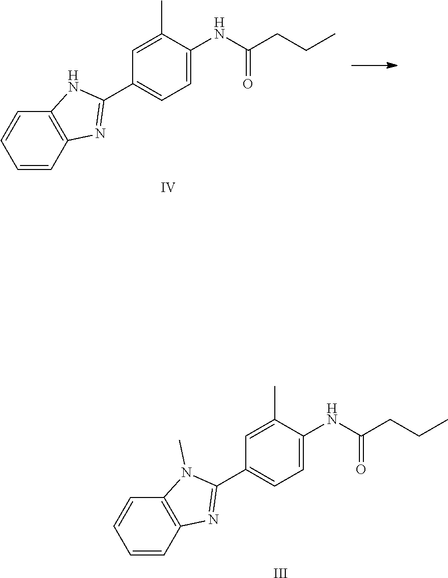 [SMILES: CCCC(=O)Nc1ccc(-c2nc3ccccc3[nH]2)cc1C ; "IV" ; CCCC(=O)Nc1ccc(-c2nc3ccccc3n2C)cc1C ; "III"]
In particular, the methylating agent is selected from the group consisting of methyl iodide, dimethyl sulfate and dimethyl carbonate;
In particular, the reaction is carried out in the presence of a basic reagent in a solvent,
In particular, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;
In particular, the solvent is one selected from the group consisting of tetrahydrofuran, dioxane, ethylene glycol dimethyl ether, methanol, ethanol, isopropanol, n-butanol, tert-butanol, ethylene glycol, acetone, acetonitrile, N,N-dimethylformamide, N,N-dimethylacetamide, N-methylpyrrolidone, dimethyl sulfoxide and water; or a mixture thereof;
Method Two: prepared by the compound of formula V-1, 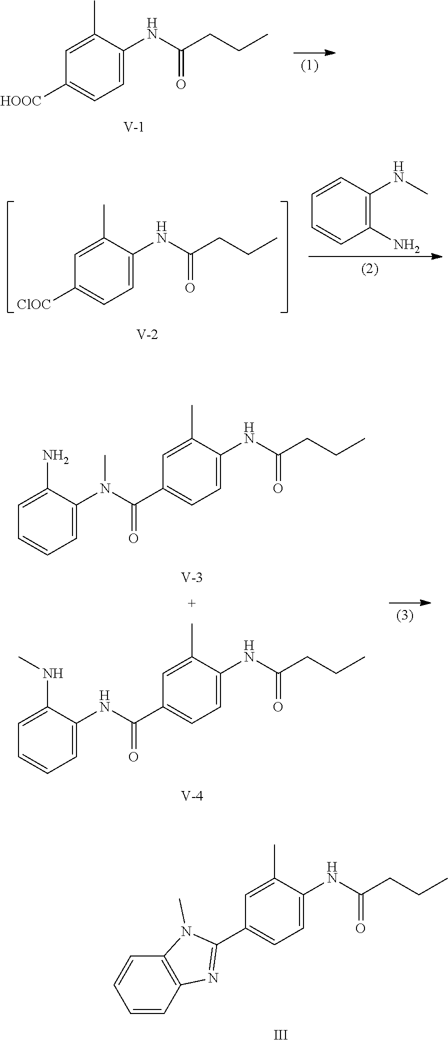
Step (1), preparing 3-methyl-4-n-butyrylaminobenzoyl chloride of formula V-2 through chlorination reaction by using 3-methyl-4-n-butyrylaminobenzoic acid as a starting material;
Step (2), reacting the compound of formula V-2 with N-methyl-o-phenylenediamine to obtain the compounds of formulae V-3 and V-4;
Step (3), conducting a condensation reaction of the compounds of formulae V-3 and V-4 in the presence of an acidic reagent, a basic reagent or a condensation reagent, to obtain the compound of formula III;
Particularly in the step (1), the chlorinating reagent used in the chlorination reaction is one selected from the group consisting of thionyl chloride, phosphorus oxychloride, phosphorus pentachloride, oxalyl chloride, phosgene, and bis(trichloromethyl) carbonate, or a mixture thereof;
Particularly in the step (1), the chlorination reaction is carried out in a solvent, in particular, the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, and methyl tert-butyl ether, or a mixture thereof; preferably is methylene chloride,
Particularly in the step (2), a basic reagent is used as an acid binding agent, in particular, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;
In particular, the step (2) is carried out in a solvent, in particular, the solvent used is one selected from the group consisting of tetrahydrofuran, dioxane, 2-methyltetrahydrofuran, ethylene glycol dimethyl ether, methyl tert-butyl ether, toluene, xylene, dichloromethane, chloroform, acetonitrile, acetone, pyridine, N,N-dimethylformamide, N-methylpyrrolidone, dimethyl sulfoxide, and water, or a mixture thereof;
In particular, the step (1) and the step (2) are carried out in one-pot method, that is, the compound of formula V-2 prepared in the step (1) is directly subjected to the reaction of the step (2) without separation;
Particularly in the step (3), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof,
Particularly in the step (3), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof;
Particularly in the step (3), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;
Particularly in the step (3), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof, alternatively, a solvent-free reaction condition is used;
Method Three: prepared by the compound of formula V-1, 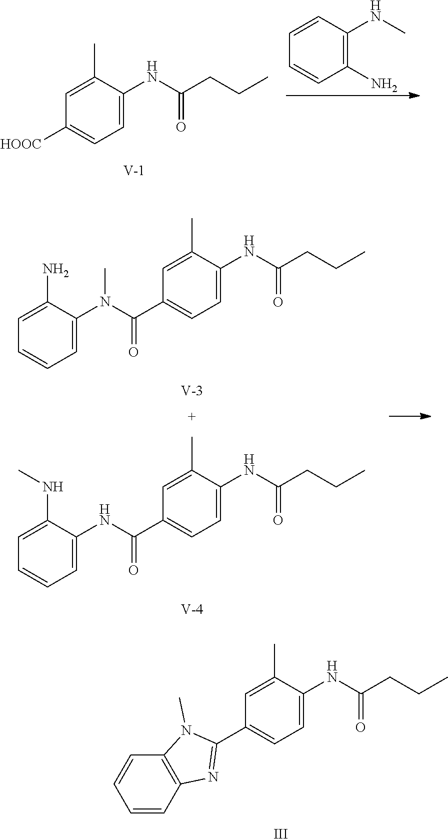
Step (1), reacting 3-methyl-4-n-butyrylaminobenzoic acid with N-methyl-o-phenylenediamine in the presence of an acidic reagent or a condensation reagent to obtain the compounds of formulae V-3 and V-4;
Step (2), reacting the compounds of formulae V-3 and V-4 in the presence of the acidic reagent or the condensation reagent in the step (1), or a basic reagent, to obtain the compound of formula III;
In particular, the step (1) and the step (2) are carried out in one-pot method, that is, in the presence of the acidic reagent or the condensation reagent, the compounds of formulae V-3 and V-4 prepared in the step (1) continually react under the reaction condition of step (1) without separation to obtain the compound of formula III;
Particularly in the step (1), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;
Particularly in the step (1), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;
Particularly in the step (2), the acidic reagent or the condensation reagent used is the same as defined in the step (1);
Particularly in the step (2), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof;
Particularly in the step (1) and the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof, alternatively, a solvent-free reaction condition is used;
Method Four: prepared by the compound of formula V-6 or V-7, 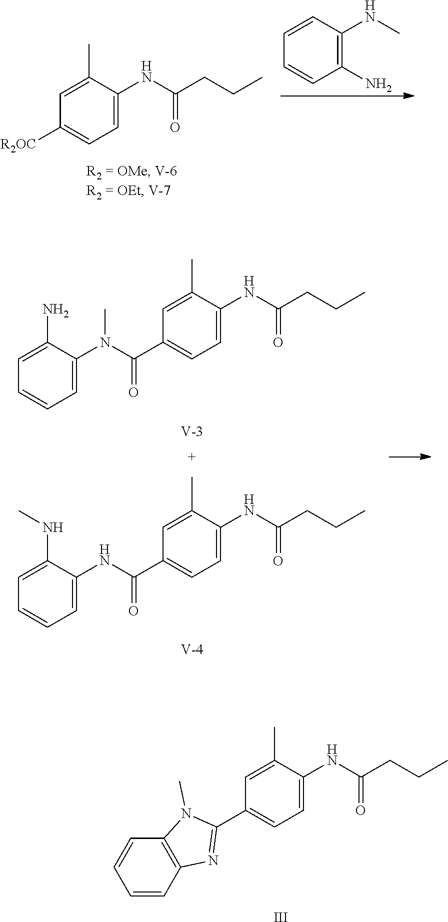
Step (1), reacting methyl 3-methyl-4-n-butyrylaminobenzoate or ethyl 3-methyl-4-n-butyrylaminobenzoate, with N-methyl-o-phenylenediamine in the presence of an acidic reagent or a basic reagent to obtain the compounds of formulae V-3 and V-4;
Step (2), reacting the compounds of formulae V-3 and V-4 in the presence of the acidic reagent or the basic reagent in the step (1), or a condensation reagent, to obtain the compound of formula III;
In particular, the step (1) and the step (2) are carried out in one-pot method, that is, in the presence of the acidic reagent or the basic reagent, the compounds of formulae V-3 and V-4 prepared in the step (1) continually react under the reaction condition of step (1) without separation to obtain the compound of formula III;
Particularly in the step (1), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;
Particularly in the step (1), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof;
Particularly in the step (2), the acidic reagent or the basic reagent used is the same as defined in the step (1);
Particularly in the step (2), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;
Particularly in the step (1) and the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof, alternatively, a solvent-free reaction condition is used;
Method Five: prepared by the compound of formula VI, 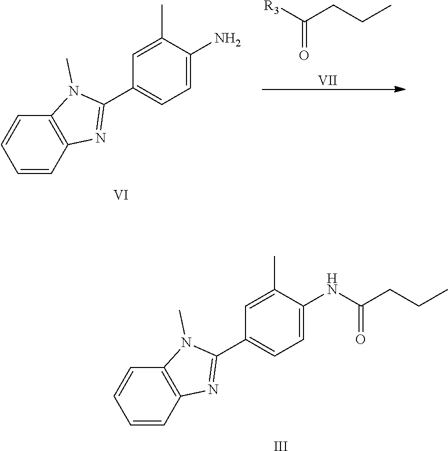
reacting 2-methyl-4-(1-methyl-1H-benzo[d]imidazol-2-yl)aniline with the compound of formula VI to obtain the compound of formula III;
Method Six: prepared by the compound of formula VIII, 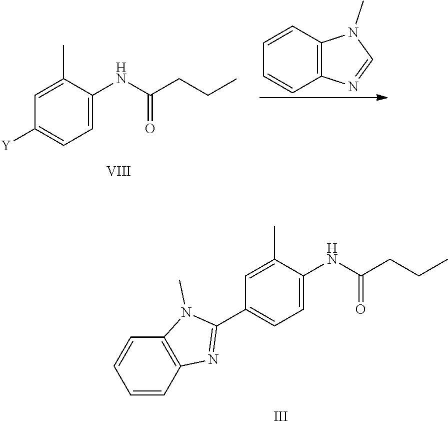
wherein, Y is Cl, Br and I,
reacting the compound of formula VIII with N-methylbenzimidazole in the presence of a basic reagent to obtain the compound of formula III,
In particular, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, sodium acetate, potassium acetate, magnesium acetate, sodium pivalate, potassium pivalate, magnesium pivalate, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;
In particular, the reaction is carried out in the presence of a transition metal compound and the complex thereof, the transition metal compound is one selected from the group consisting of cuprous chloride, cuprous bromide, cuprous iodide, cuprous oxide, cuprous cyanide, cuprous acetate, copper chloride, copper bromide, copper oxide, copper acetate, copper sulfate, copper nitrate, palladium chloride, palladium acetate, palladium trifluoroacetate, palladium trifluoromethanesulfonate, bis(dibenzylideneacetone)palladium, tris(dibenzylideneacetone)dipalladium, tetrakis(triphenylphosphine)palladium, nickel dichloride, nickel acetate, bis(acetylacetone)nickel, nickel trifluoroacetate, nickel trifluoromethanesulfonate, and bis(1,5-cyclooctadiene)nickel, or a mixture thereof; the ligand used for the transition metal compound is one selected from the group consisting of ethylenediamine, N-methylethylenediamine, N-butyl ethylenediamine, N,N′-dimethylethylenediamine, N,N-dimethylethylenediamine, trimethylethylenediamine, tetramethylethylenediamine, (cis)-1,2-cyclohexanediamine, (trans)-1,2-cyclohexanediamine, 1,2-cyclohexanediamine racemate, (trans)-N,N′-dimethyl-1,2-cyclohexanediamine, (trans)-N,N′-diethyl-1,2-cyclohexanediamine, (trans)-N,N′-diisopropyl-1,2-cyclohexanediamine, 2,2′-bipyridine, 1,10-phenanthroline, 2,9-dimethyl-1,10-phenanthroline, 3,4,7, 8-tetramethyl-1,10-phenanthroline, 4,7-diphenyl-1,10-phenanthroline, triphenylphosphine, tricyclohexylphosphine, tri-tert-butylphosphine, 1,2-bis(diphenylphosphine)ethane, 1,2-bis(diphenylphosphine)propane, 1,1′-bis(di-phenylphosphine)ferrocene, 4,5-bis(diphenylphosphine)-9,9-dimethylxanthene and 1,1′-binaphthyl-2,2′-bisdiphenylphosphine, or a mixture thereof,
In particular, the reaction is carried out in a solvent, the solvent is one selected from the group consisting of dioxane, 2-methyltetrahydrofuran, ethylene glycol dimethyl ether, toluene, xylene, acetonitrile, acetone, ethanol, isopropanol, n-butanol, tert-butanol, ethylene glycol, pyridine, N,N-dimethylformamide, N,N-dimethylacetamide, N-methylpyrrolidone, dimethyl sulfoxide, and water, or a mixture thereof.

3. The method according to claim 2, wherein, in method Five, the compound of formula VI is prepared by the following method: 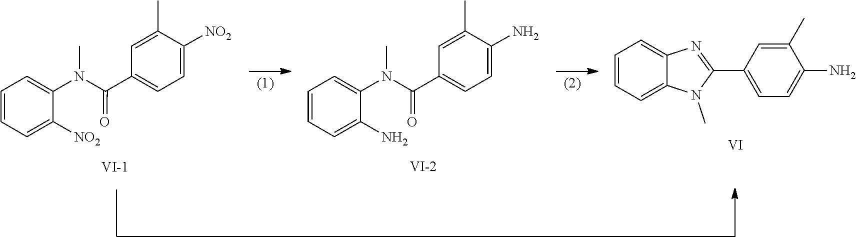
Step (1), obtaining the compound of formula VI-2 from the compound of formula VI-1 under reducing conditions;
Step (2), obtaining the compound of formula VI from the compound of formula VI-2 in the presence of an acidic reagent, a basic reagent, or a condensation reagent;
Alternatively, obtaining directly the compound of formula VI from the compound of formula VI-1 by adding an acidic reagent or a basic reagent in the reduction system of step (1); in particular, the acidic reagent or the basic reagent used in the reaction process is the same as defined in the step (1) of the method Four in claim 2;
Particularly in the step (1), the reducing agent used in the reducing conditions is a nitro reducing agent, which is selected from the group consisting of hydrogen, metal reducing agents, metal chlorides, complex hydrides, sulfur-containing reducing agents, and so on, wherein the hydrogen reduction is conducted with the addition of a catalyst, the catalyst used is selected from the group consisting of Cu, Ni, Pd, Pt, Ru, Rh and their oxides, hydroxides, chlorides, complexes formed with carbon or corresponding organometallic complexes; the metal reducing agent is selected from the group consisting of iron powder and zinc powder; the metal chloride is selected from the group consisting of stannous chloride dihydrate, and titanium trichloride; the complex hydride is selected from the group consisting of lithium aluminum hydride; the sulfur-containing reducing agent is selected from the group consisting of sodium hydrosulfide, sodium sulfide, ammonium sulfide, sodium sulfite, sodium bisulfite, and sodium dithionite;
In particular, the step (1) is carried out in a solvent, the solvent is one selected from the group consisting of methanol, ethanol, propanol, isopropanol, n-butanol, tert-butanol, ethylene glycol, dioxane, 2-methyl tetrahydrofuran, ethylene glycol dimethyl ether, toluene, xylene, acetone, ethyl acetate, n-butyl acetate, N,N-dimethylformamide, N,N-dimethylacetamide, dimethylsulfoxide, formic acid, acetic acid, hydrochloric acid, sulfuric acid and water, or a mixture thereof;
In particular, the acidic reagent, the basic reagent or the condensation reagent in the step (2) is the same as defined in the step (2) of method Four in claim 2;
In particular, in the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof, alternatively, a solvent-free reaction condition is used.

4. The method according to claim 2, wherein the method Five is conducted according to one of the following three methods:
Method (a), when R₃ is chlorine, or bromine, or n-butyryloxy, reacting the compound of formula VI with n-butyryl chloride, or n-butyryl bromide, or n-butyric anhydride to prepare the compound of formula III,
In particular, method (a) is conducted in presence of a basic reagent, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;
In particular, method (a) is carried out in a solvent, in particular, the solvent used is one selected from the group consisting of tetrahydrofuran, dioxane, 2-methyltetrahydrofuran, ethylene glycol dimethyl ether, methyl tert-butyl ether, toluene, xylene, dichloromethane, chloroform, acetonitrile, acetone, pyridine, N,N-dimethylformamide, N-methylpyrrolidone, dimethyl sulfoxide, and water, or a mixture thereof;
Method (b), when R₃ is methoxy or ethoxy, reacting the compound of formula VI with methyl n-butyrate or ethyl n-butyrate to prepare the compound of formula III,
In particular, method (b) is carried out in the presence of an acidic reagent or a basic reagent,
Particularly in method (b), the acidic reagent used is one selected from the group consisting of hydrochloric acid, sulfuric acid, nitric acid, phosphoric acid, polyphosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof,
Particularly in the step (b), when using an acidic reagent, the reaction is carried out in a solvent or using solvent-free reaction conditions, in particular, when using an acidic reagent, the solvent used is one selected from the group consisting of dichloromethane, chloroform, benzene, toluene, xylene, methanol, ethanol, isopropanol, n-butanol, tert-butanol, and ethylene glycol, or a mixture thereof,
Particularly in method (b), the basic reagent used is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof, preferably is sodium methoxide,
Particularly in the method (b), when using a basic reagent, the reaction is carried out in a solvent, in particular, when using a basic reagent, the solvent used is one selected from the group consisting of dichloromethane, chloroform, benzene, toluene, xylene, methanol, ethanol, isopropanol, n-butanol, tert-butanol, ethylene glycol, N,N-dimethylformamide, N,N-dimethylacetamide, and N-methylpyrrolidone, or a mixture thereof;
Method (c), when R₃ is hydroxyl, reacting the compound of formula VI with n-butyric acid in the presence of an acidic reagent or a condensation reagent to prepare the compound of formula III,
Particularly in method (c), the acidic reagent used is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof,
Particularly in method (c), when using an acidic reagent, the reaction is carried out in a solvent or using solvent-free reaction conditions, in particular, when using an acidic reagent, the solvent is one selected from the group consisting of dichloromethane, chloroform, benzene, toluene, xylene, methanol, ethanol, isopropanol, n-butanol, tert-butanol, and ethylene glycol, or a mixture thereof,
Particularly in method (c), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;
Particularly in the method (c), when using a condensation reagent, the reaction is carried out in a solvent, in particular, when using a condensation reagent, the solvent used is one selected from the group consisting of dichloromethane, chloroform, benzene, toluene, xylene, tetrahydrofuran, dioxane, ethylene glycol dimethyl ether, acetone, acetonitrile, N,N-dimethylformamide, N,N-dimethylacetamide, and N-methylpyrrolidone, or a mixture thereof.

5. The method according to claim 2, wherein the compound of formula IV is prepared by one of the following methods:
Method I: prepared by the compound of formula V-1, 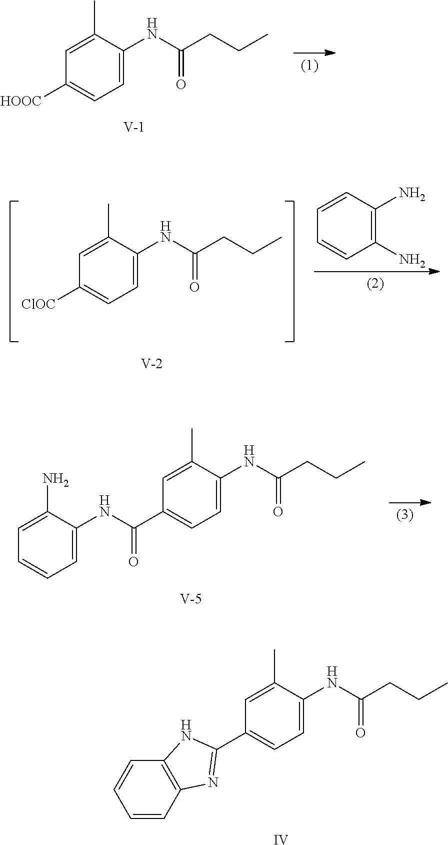
Step (1), preparing 3-methyl-4-n-butyrylaminobenzoyl chloride through chlorination reaction by using 3-methyl-4-n-butyrylaminobenzoic acid as a starting material;
Step (2), reacting the produced compound of formula V-2 with o-phenylenediamine to obtain the compound of formula V-5;
Step (3), conducting a condensation reaction of the compound of formula V-5 in the presence of an acidic reagent, a basic reagent or a condensation reagent, to obtain the compound of formula IV;
Particularly in the step (1), the chlorinating reagent used in the chlorination reaction is one selected from the group consisting of thionyl chloride, phosphorus oxychloride, phosphorus pentachloride, oxalyl chloride, phosgene, and bis(trichloromethyl) carbonate, or a mixture thereof;
Particularly in the step (1), the chlorination reaction is carried out in a solvent, in particular, the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, and methyl tert-butyl ether, or a mixture thereof; preferably is methylene chloride;
Particularly in the step (2), a basic reagent is used as an acid binding agent, in particular, the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;
In particular, the step (2) is carried out in a solvent, in particular, the solvent used is one selected from the group consisting of tetrahydrofuran, dioxane, 2-methyltetrahydrofuran, ethylene glycol dimethyl ether, methyl tert-butyl ether, toluene, xylene, dichloromethane, chloroform, acetonitrile, acetone, pyridine, N,N-dimethylformamide, N-methylpyrrolidone, dimethyl sulfoxide, and water, or a mixture thereof;
In particular, the step (1) and the step (2) are carried out in one-pot method, that is, the compound of formula V-2 prepared in the step (1) is directly subjected to the reaction of the step (2) without separation;
Particularly in step (3), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof,
Particularly in the step (3), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof;
Particularly in the step (3), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;
Particularly in the step (3), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof, alternatively, a solvent-free reaction condition is used;
Method II: prepared by the compound of formula V-1, 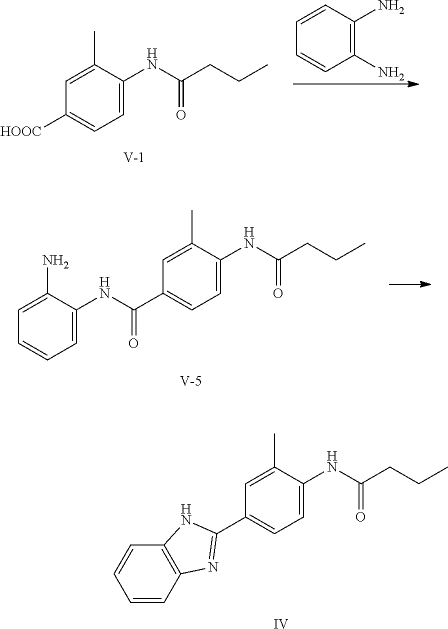
Step (1), reacting 3-methyl-4-n-butyrylaminobenzoic acid with o-phenylenediamine in the presence of an acidic reagent or a condensation reagent to obtain the compound of formula V-5;
Step (2), reacting the compound of formula V-5 in the presence of the acidic reagent or the condensation reagent in the step (1), or a basic reagent, to obtain the compound of formula IV;
In particular, the step (1) and the step (2) are carried out in one-pot method, that is, the compound of formula V-5 prepared in the step (1) continually reacts under the reaction condition of step (1) without separation to obtain the compound of formula IV;
Particularly in the step (1), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;
Particularly in the step (1), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;
Particularly in the step (2), the acidic reagent or the condensation reagent used is the same as defined in the step (1);
Particularly in the step (2), the basic reagent used is one selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof;
Particularly in the step (1) and the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof, alternatively, a solvent-free reaction condition is used;
Method III: 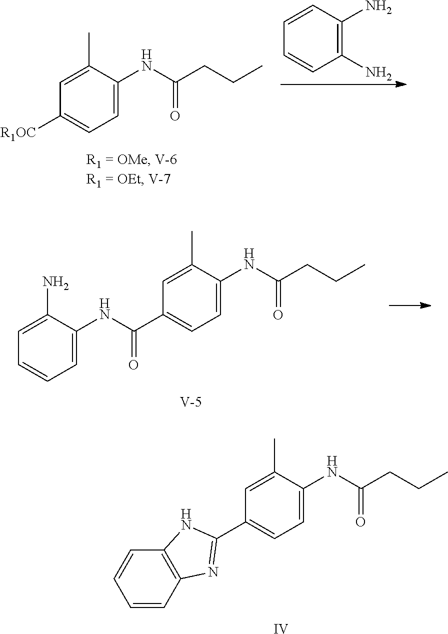
Step (1), reacting methyl 3-methyl-4-n-butyrylaminobenzoate or ethyl 3-methyl-4-n-butyrylaminobenzoate, with o-phenylenediamine in the presence of an acidic reagent or a basic reagent to obtain the compound of formula V-5;
Step (2), reacting the compound of formula V-5 in the presence of the acidic reagent or the basic reagent in the step (1), to obtain the compound of formula IV;
In particular, the step (1) and the step (2) are carried out in one-pot method, that is, in the presence of the acidic reagent or the basic reagent, the compound of formula V-5 prepared in the step (1) continually reacts under the reaction condition of step (1) without separation to obtain the compound of formula IV;
Particularly in the step (1), the acidic reagent used is one selected from the group consisting of conventional inorganic protonic acids, organic carboxylic acids, organic sulfonic acids, organic phosphoric acids, organic Lewis acids, and inorganic Lewis acids, or a mixture thereof; preferably is one selected from the group consisting of hydrochloric acid, sulfuric acid, nitric acid, phosphoric acid, hydrobromic acid, hydrofluoric acid, acetic acid, propionic acid, trifluoroacetic acid, malonic acid, benzoic acid, nitrobenzoic acid, methanesulfonic acid, p-toluenesulfonic acid, boric acid, boron trifluoride, boron tribromide, boron trichloride, aluminum trichloride, trimethyl aluminum, ferric chloride, zinc dichloride, indium trichloride, and titanium tetrachloride, or a mixture thereof;
Particularly in the step (1), the basic reagent used is selected from the group consisting of alkali metal organic bases, alkaline earth metal organic bases, and alkali metal fluorides, preferably is one selected from the group consisting of lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, lithium fluoride, sodium fluoride, potassium fluoride, and cesium fluoride, or a mixture thereof;
Particularly in the step (2), the acidic reagent or the basic reagent used is the same as defined in the step (1);
Particularly in the step (2), the condensation reagent used is one selected from the group consisting of concentrated sulfuric acid, polyphosphoric acid, 4,5-dicyanoimidazole, N,N′-carbonyldiimidazole, dicyclohexylcarbodiimide, diisopropylcarbodiimide, 1-(3-dimethylaminopropyl)-3-ethylcarbodiimide, 1-hydroxybenzotriazole, O-(7-azabenzotriazole-1-yl)-bis(dimethylamino)carbonium hexafluorophosphate, O-(benzotriazole-1-yl)-bis(dimethylamino)carbonium hexafluorophosphate, orthoformic acid triester, orthoformic acid tetraester, orthoacetic acid triester and orthoacetic acid tetraester, or a mixture thereof;
In particular, in the step (1) and the step (2), the solvent used is one selected from the group consisting of dichloromethane, chloroform, benzene, toluene, xylene, chlorobenzene, methanol, ethanol, isopropanol, n-butanol, tert-butanol, ethylene glycol, tetrahydrofuran, dioxane, ethylene glycol dimethyl ether, acetone, acetonitrile, N,N-dimethylformamide, N,N-dimethylacetamide, and N-methylpyrrolidone, or a mixture thereof, alternatively, a solvent-free reaction condition is used.

6. The method according to claim 3, wherein the compound of formula VI-1 is prepared by one of the following methods:
Method IV: 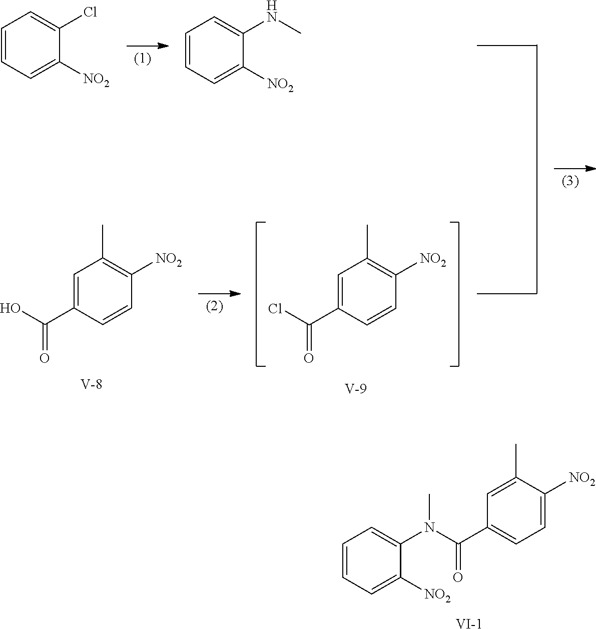
Step (1), reacting o-nitrochlorobenzene in a methylamine aqueous solution under heating conditions to obtain o-nitrobenzylamine;
Step (2), conducting a chlorination reaction of the compound of formula V-8 to obtain the compound of formula V-9;
Step (3), reacting the compound of formula V-9 with o-nitrobenzylamine to obtain the compound of formula VI-1;
Particularly in the step (1), the concentration of the methylamine aqueous solution is 20-30%, and the reaction is carried out at 100-150° C. and 3-10 atm;
In particular, the step (2) and the step (3) are carried out in a solvent;
In particular, the step (2) and the step (3) are carried out in one-pot method, that is, the compound of formula V-9 prepared in the step (2) is directly subjected to the reaction of the step (3) without separation;
wherein, the chlorinating reagent used in the chlorination reaction, the basic reagent and the solvent in the steps (2) and (3) of the reaction process are the same as defined for the chlorinating reagent used in the chlorination reaction, the basic reagent and the solvent in the steps (1) and (2) of the method I in claim 5;
Method V: 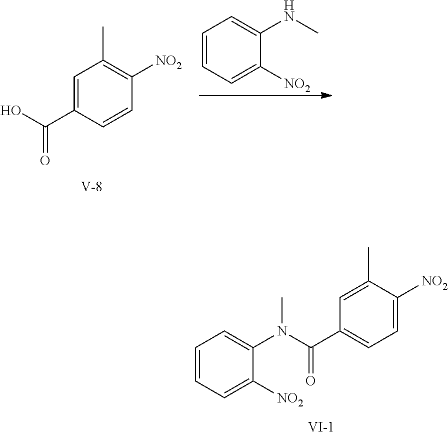
reacting the compound of formula V-8 with o-nitrobenzylamine in the presence of an acidic reagent or a condensation reagent in a solvent to obtain the compound of formula VI-1;
In particular, the acidic reagent, the condensation reagent and the solvent in the reaction process are the same as defined for the acidic reagent, the condensation reagent and the solvent used in the step (1) of the method II in claim 5;
Method VI: 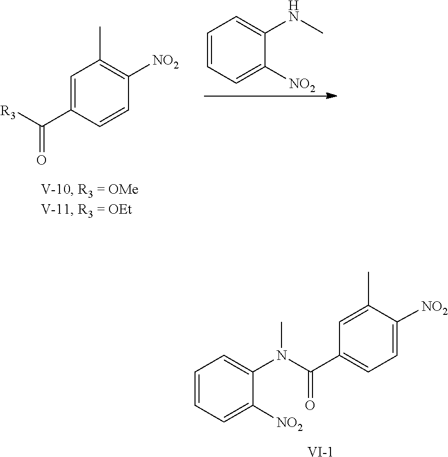
reacting the compound of formula V-10 or the compound of formula V-11 with o-nitrobenzylamine in the presence of an acidic reagent or a basic reagent in a solvent to obtain the compound of formula VI-1;
In particular, the acidic reagent, the basic reagent and the solvent in the reaction process are the same as defined for the acidic reagent, the basic reagent and the solvent in the step (1) of the method III in claim 5;
Method VII: 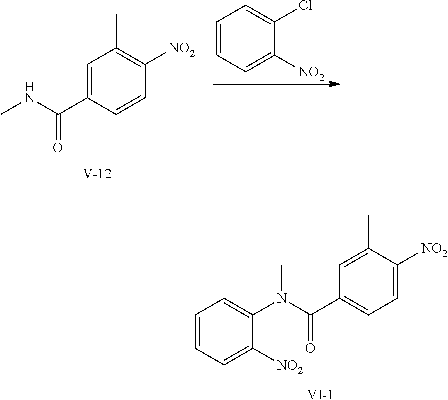
reacting the compound of formula V-12 with o-nitrochlorobenzene in the presence of a basic reagent, a transition metal compound and its complex in a solvent to obtain the compound of formula VI-1;
In particular, the basic reagent, the transition metal compound and its complex, and the solvent in the reaction process are the same as defined for the basic reagent, the transition metal compound and its complex, and the solvent in the method Six in claim 2;
Method VIII: 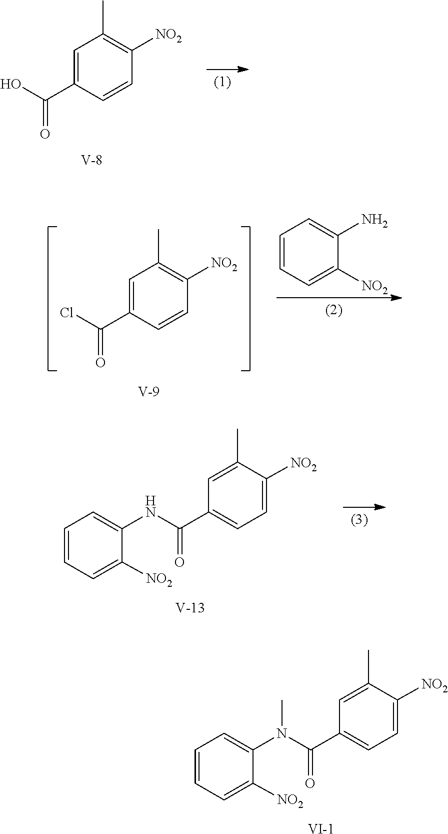
Step (1), preparing 3-methyl-4-nitrobenzoyl chloride through chlorination reaction with 3-methyl-4-nitrobenzoic acid as a starting material;
Step (2), reacting the produced compound of formula V-9 with o-nitrobenzylamine in the presence of a basic reagent to obtain the compound of formula V-13;
Step (3), reacting the compound of formula V-13 with a methylating reagent to prepare the compound of formula VI-1;
In particular, the step (1) and the step (2) are carried out in a solvent;
In particular, the step (1) and the step (2) are carried out in one-pot method, that is, the compound of formula V-9 prepared in the step (1) is directly subjected to the reaction of the step (2) without separation;
wherein, the chlorinating reagent used in the chlorination reaction, the basic reagent and the solvent in the steps (1) and (2) of the reaction process are the same as defined for the chlorinating reagent used in the chlorination reaction, the basic reagent and the solvent in the steps (1) and (2) of the method I in claim 5,
Particularly in the step (3), the methylating agent is selected from the group consisting of methyl iodide, methyl chloride, dimethyl sulfate and dimethyl carbonate;
Particularly in the step (3), the reaction is carried out in the presence of a basic reagent in a solvent,
Particularly in the step (3), the basic reagent is one selected from the group consisting of lithium carbonate, sodium carbonate, potassium carbonate, cesium carbonate, sodium bicarbonate, potassium bicarbonate, sodium phosphate, potassium phosphate, sodium monohydrogen phosphate, potassium monohydrogen phosphate, lithium hydroxide, sodium hydroxide, potassium hydroxide, magnesium carbonate, magnesium hydroxide, calcium carbonate, calcium hydroxide, calcium oxide, magnesium oxide, lithium methoxide, sodium methoxide, potassium methoxide, sodium ethoxide, potassium ethoxide, lithium isopropoxide, sodium isopropoxide, potassium isopropoxide, lithium tert-butoxide, sodium tert-butoxide, potassium tert-butoxide, magnesium methoxide, magnesium ethoxide, magnesium tert-butoxide, ammonia water, triethylamine, diisopropylamine, diisopropyl ethyl amine, tri-n-butyl amine, pyridine, 2-methylpyridine, 2,6-dimethylpyridine, 4-dimethylaminopyridine, tetrahydropyrrole, morpholine, piperidine and 2,2,6,6-tetramethylpiperidine; or a mixture thereof;
Particularly in the step (3), the solvent is one selected from the group consisting of tetrahydrofuran, dioxane, ethylene glycol dimethyl ether, methanol, ethanol, isopropanol, n-butanol, tert-butanol, ethylene glycol, acetone, acetonitrile, N,N-dimethylformamide, N,N-dimethylacetamide, N-methylpyrrolidone, dimethyl sulfoxide and water; or a mixture thereof

7. An intermediate or a salt thereof for preparing the compound of formula III, wherein the intermediate is selected from the group consisting of the compounds of formulae IV, V-3, V-4, V-5, and VI-1:

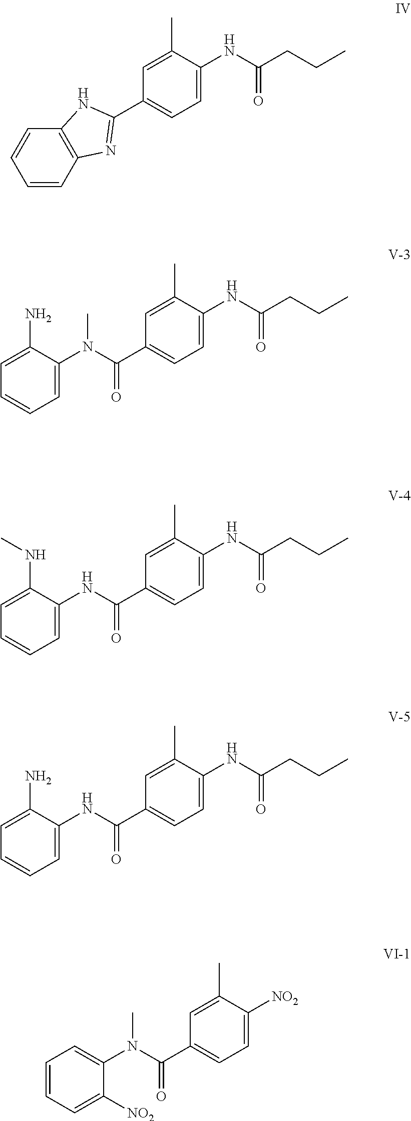 [SMILES: CCCC(=O)Nc1ccc(-c2nc3ccccc3[nH]2)cc1C ; CCCC(=O)Nc1ccc(C(=O)N(C)c2ccccc2N)cc1C ; CCCC(=O)Nc1ccc(C(=O)Nc2ccccc2NC)cc1C ; CCCC(=O)Nc1ccc(C(=O)Nc2ccccc2N)cc1C ; Cc1cc(C(=O)N(C)c2ccccc2[N+](=O)[O-])ccc1[N+](=O)[O-]]

8. A method for preparing the compound of formula II, comprising: 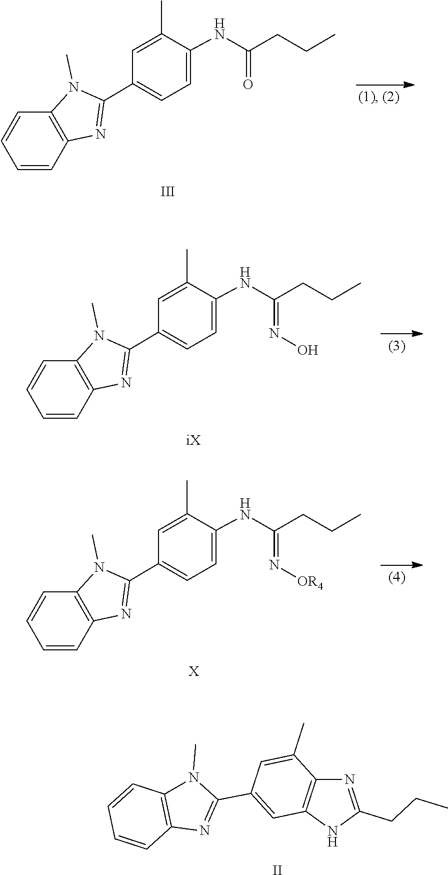
Step (1), reacting the compound of formula III with a chlorinating reagent;
Step (2), reacting the chlorination reaction mixture obtained in the step (1) with a hydroxylamine reagent to obtain the compound of formula IX;
Step (3), reacting the compound of formula IX with an acid chloride or acid anhydride reagent to obtain a compound of formula X;
Step (4), reacting the compound of formula X in the presence of a basic reagent to obtain the compound of formula II;
Particularly in the step (1), the chlorinating reagent used is one selected from the group consisting of thionyl chloride, phosphorus oxychloride, phosphorus pentachloride, oxalyl chloride, phosgene, and bis(trichloromethyl) carbonate, or a mixture thereof; the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, and methyl tert-butyl ether, or a mixture thereof;
Particularly in the step (2), the hydroxylamine reagent used is selected from the group consisting of a free base-type hydroxylamine, hydroxylamine hydrochloride, or a salt formed by hydroxylamine;
Particularly in the step (3), in the compound of formula X, R₄ is a carboxylic acid acyl of —C(═O)R₅, a sulfonyl of —SO₂R₆, an alkoxycarbonyl of —C(═O)—OR₇, an alkylaminocarbonyl of —C(═O)—NR₈R_{9,} or an alkoxyphosphoryl of —P(═O)(OR₁₀)₂;
wherein, R₅ to R₁₀ are each independently hydrogen, substituted or unsubstituted C₁-C₂₀ linear or branched or cyclic alkyl, substituted or unsubstituted C₁-C₂₀ linear or branched or cyclic alkenyl, substituted or unsubstituted benzyl, substituted or unsubstituted C₆-C₂₀ aryl; in the case that R₅ to R₁₀ are substituted C₁-C₂₀ linear or branched or cyclic alkyl, substituted C₁-C₂₀ linear or branched or cyclic alkenyl, substituted benzyl, or substituted C₆-C₂₀ aryl, the substituent is selected from the group consisting of cyano, nitro, amino, hydroxyl, mercapto, halogen, phenyl, C₁-C₂₀ linear or branched or cyclic alkyl, C₁-C₂₀ linear or branched or cyclic alkenyl, and C₁-C₂₀ linear or branched or cyclic alkoxy;
Particularly in the step (3), the acid chloride or acid anhydride reagent used is an acid chloride or acid anhydride corresponding to R₄, preferably is one selected from the group consisting of acetyl chloride, trifluoroacetyl chloride, benzoyl chloride, p-nitrobenzoyl chloride, p-chlorobenzoyl chloride, methanesulfonyl chloride, trifluoromethanesulfonyl chloride, p-toluenesulfonyl chloride, acetic anhydride, trifluoroacetic anhydride, benzoic anhydride, p-nitrobenzoic anhydride, p-chlorobenzoic anhydride, methanesulfonic anhydride, trifluoromethanesulfonic anhydride, p-toluenesulfonic anhydride, methyl chloroformate, ethyl chloroformate, benzyl chloroformate, di-tert-butyl dicarbonate, N,N-dimethylchloroformamide, diethoxyphosphoryl chloride, and so on, or a mixture thereof;
Particularly in the step (3), the reaction is carried out in the presence of a basic reagent, the basic reagent is one selected from the group consisting of lithium carbonate, lithium hydroxide, lithium tert-butoxide, sodium carbonate, sodium bicarbonate, sodium hydroxide, sodium phosphate, sodium methoxide, sodium ethoxide, sodium isopropoxide, sodium tert-butoxide, potassium carbonate, potassium bicarbonate, potassium hydroxide, potassium phosphate, potassium methoxide, potassium ethoxide, potassium tert-butoxide, cesium carbonate, cesium hydroxide, magnesium carbonate, magnesium hydroxide, magnesium phosphate, magnesium oxide, magnesium methoxide, magnesium ethoxide, magnesium isopropoxide, magnesium tert-butoxide, triethylamine, diisopropylamine, diisopropylethylamine, tri-n-butylamine, pyridine, 2-methylpyridine, 2,6-dimethylpyridine, 4-dimethylaminopyridine, tetrahydropyrrole, morpholine, piperidine, and 2,2,6,6-tetramethylpiperidine; or a mixture thereof;
Particularly in the step (3), the reaction is carried out in a solvent, the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, methyl tert-butyl ether and water, or a mixture thereof;
Particularly in the step (4), the basic reagent used is one selected from the group consisting of lithium carbonate, lithium hydroxide, lithium tert-butoxide, sodium carbonate, sodium bicarbonate, sodium hydroxide, sodium phosphate, sodium methoxide, sodium ethoxide, sodium isopropoxide, sodium tert-butoxide, potassium carbonate, potassium bicarbonate, potassium hydroxide, potassium phosphate, potassium methoxide, potassium ethoxide, potassium tert-butoxide, cesium carbonate, cesium hydroxide, magnesium carbonate, magnesium hydroxide, magnesium phosphate, magnesium oxide, magnesium methoxide, magnesium ethoxide, magnesium isopropoxide, magnesium tert-butoxide, triethylamine, diisopropylamine, diisopropylethylamine, tri-n-butylamine, pyridine, 2-methyl pyridine, 2,6-demethylpyridine, 4-dimethylaminopyridine, tetrahydropyrrole, morpholine, piperidine, and 2,2,6,6-tetramethylpiperidine, or a mixture thereof;
Particularly in the step (4), the solvent used is one selected from the group consisting of dichloromethane, chloroform, benzene, toluene, xylene, chlorobenzene, acetonitrile, tetrahydrofuran, 2-methyltetrahydrofuran, dioxane, ethylene glycol dimethyl ether, methyl tert-butyl ether, and water, or a mixture thereof.

9. The method according to claim 8, wherein the step (3) and the step (4) are performed in one-pot method, or the step (1) to the step (4) are performed in one-pot method.

10. An intermediate or a salt thereof for preparing the compound of formula II, wherein the intermediate is selected from the group consisting of the compounds of formulae IX and X, or salts thereof: 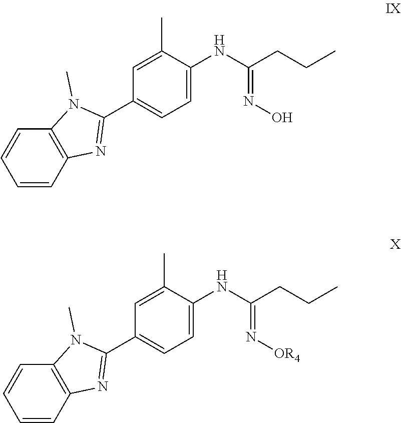
wherein, R₄ in the compound of formula IX is the same as defined in claim 8, is —C(═O)R₅, —SO₂R₆, —C(═O)—OR₇, —C(═O)—NR₈R₉, or —P(═O)(OR₁₀)₂
wherein, R₅ to R₁₀ are each independently hydrogen, substituted or unsubstituted C₁-C₂₀ linear or branched or cyclic alkyl, substituted or unsubstituted C₁-C₂₀ linear or branched or cyclic alkenyl, substituted or unsubstituted benzyl, substituted or unsubstituted C₆-C₂₀ aryl; in the case that R₅ to R₁₀ are substituted C₁-C₂₀ linear or branched or cyclic alkyl, substituted C₁-C₂₀ linear or branched or cyclic alkenyl, substituted benzyl, or substituted C₆-C₂₀ aryl, the substituent is selected from the group consisting of cyano, nitro, amino, hydroxyl, mercapto, halogen, phenyl, C₁-C₂₀ linear or branched or cyclic alkyl, C₁-C₂₀ linear or branched or cyclic alkenyl, and C₁-C₂₀ linear or branched or cyclic alkoxy.
